(19) United States
(12) Patent Application Publication
(10) Pub. No.: US 2006/0078658 A1
(43) Pub. Date: Apr. 13, 2006

(54) Food quality sensor and methods thereof

(76) Inventors: Megan M. Owens, Waltham, MA (US); Marco A. Bonne, Carlisle, MA (US); Tai M. Chan, Brookline, MA (US); John R. Williams, Lexington, MA (US)

Correspondence Address: MINTZ LEVIN COHN FERRIS GLOVSKY & POPEO, 666 THIRD AVENUE, NEW YORK, NY, 10017, US

(21) Appl. No.: 11/243,941

(22) Filed: Oct. 4, 2005

Related U.S. Application Data
(60) Provisional application No. 60/615,912, filed on Oct. 4, 2004.

(51) Int. Cl. G01N 33/02

(52) U.S. Cl. 426/231

(57) ABSTRACT

Embodiments of the present invention are directed to methods and devices for determining freshness of food products, and materials and devices related thereto. In some embodiments, a sensor device is provided which may include an ergonomic body including a slot for receiving a sensor card, a microcontroller, a plurality of LEDs for displaying operational status of the sensor device and/or displaying a result and audio means for audibly presenting operation status of the sensor device and/or audibly presenting a result. The device may also include a control program operating on the microcontroller for operating the sensor device and for determining a freshness of food, a first air inlet for placement proximate to a food product to receive air from around a food product for testing and a replaceable sensor card including a plurality of sensors an a sensor card inlet and a sensor card outlet.

### CLAIM TO PRIORITY

The subject application claims priority under 35 U.S.C. §1.119(e) of U.S. provisional patent application Ser. No. 60/615,912, filed Oct. 4, 2004, the entire disclosure of which is herein incorporated by reference.

### FIELD OF THE INVENTION

The invention relates generally to food and beverage sensors, and more particularly to methods and devices for determining the perishable state of food or beverages (together hereinafter referred to as “food product(s)”).

### BACKGROUND OF THE INVENTION

Many articles of commerce, such as food, are perishable items (a perishable(s)). When a perishable is enclosed in packaging, it may not be readily apparent when the article has exceeded its useful lifetime. Accordingly, determining the perishable state of food is a critical task throughout food production, storage, distribution, and consumption/purchase. Many food products are subject to spoilage, either as a result of improper handling (e.g., poultry or meat being exposed to excessive temperatures during transit), or simply due to aging (spoilage is inevitable).

Today, food distributors typically label their products with expiration dates/codes, but these dates essentially only represent an estimate—that is, they assume an average, or even perfect, “heat history” that corresponds to a known aging profile. Food distributors generally do not continuously monitor the quality of their products, and thus, some spoiled food may make it through the supply chain to a retail store to be purchased by shoppers. Spoiled food not only poses risks due to illness, but also represents lost revenue for grocers and squandered wages for the consumer. Moreover, “fresh” or still good quality food products may be discarded too early (i.e., before they are actually spoiled), which is both a waste of product and money.

Although devices currently exist for determining the perishable state of food, such devices do not provide a quick, simple, and effective diagnostic, since such devices:
may use harmful substances as the indicator of spoilage
utilize a generic indicator that is not “tuned” to the particular food being detected (levels that would indicate spoilage in some foods may be perfectly consistent with freshness in other foods; e.g., fish, chicken, beef, pork), or
requiring too long a time period and costly (e.g., running bacteriology colony tests in a lab).

### SUMMARY OF THE INVENTION

Embodiments of the present invention address the above-noted needs in the industry and provide a simple, reliable food product spoilage detector, which is preferably handheld, that preferably offers rapid response time and may be optionally tunable for variations in foods and contaminants.

In addition, the present invention includes particularly advantageous embodiments, in particular, a handheld spoilage detector which provides one-handed operation, allowing an operator to easily select the type of food to be analyzed and one which may also provide a visual and/or audio indication whether the food is fresh, good (not fresh, but not yet spoiled and safe to eat), or spoiled.

Accordingly, in one embodiment of the present invention, a sensor device is provided which may include a body, an air inlet for placement proximate to a food product for sampling air, an air outlet and at least one sensor. An electrical property of the sensor varies upon exposure of the sensor to at least one of a plurality of predetermined molecules, particles, bacteria, viruses and biological cells from air flowed across the at least one sensor. The device may also include an air pump for drawing air from the inlet and across the at least one sensor.

In yet another embodiment of the present invention, a sensor device is provided which may include an ergonomic body having a slot for receiving a sensor card, a microcontroller, a plurality of LEDs for displaying an operational status of the sensor device and/or displaying a result and audio means for audibly presenting operation status of the sensor device and/or audibly presenting a result. The device may also include a control program operating on the microcontroller for operating the sensor device and for determining a freshness of food, a first air inlet for placement proximate to a food product to receive air from around a food product for testing and a replaceable sensor card having a plurality of sensors, a sensor card inlet and a sensor card outlet. The sensor card receives air in the sensor card inlet from the first air inlet, and after the air impinges on the sensors, it is vented out of the sensor card via the sensor card outlet. One or more electrical properties (e.g., resistance, capacitance) of each sensor varies upon exposure of the sensor to at least one of a plurality of predetermined molecules, particles, bacteria, viruses and biological cells contained in the air flow. The sensor device may also include a fan assembly including a manifold having an outlet for exhausting air therefrom and/or the sensor device, a fan rotor and a cover. The cover includes a fan assembly inlet which receives air from the sensor card outlet.

In yet another embodiment of the present invention, a replaceable sensor card for a sensor device is provided and may include a plurality of sensors and a plurality of electrical contacts in connection with the plurality of sensors. The contacts are received in a corresponding electrical connector in a sensor device. The sensor card includes a sensor card inlet and a sensor card outlet. The sensor card receives air into the sensor card via the inlet, where it then impinges on the sensors. The air eventually is vented out of the sensor card via sensor card outlet. An electrical property (e.g., resistance, capacitance) of each sensor varies upon exposure of the sensor to at least one of a plurality of predetermined molecules, particles, bacteria, viruses and biological cells contained in the air flow.

In another embodiment of the invention, a method for determining freshness of a food product is provided and may include measuring a first value of one or more electrical properties of one or more sensors in a food sensor device prior to the one or more sensors being exposed to an air flow received from adjacent a food product for testing, exposing the one or more of the sensors to a the air flow, measuring a second value of the one or more electrical properties of the one or more sensors and determining if the second value is greater, by a predetermined amount, than the first value.

In another embodiment of the present invention, a method for determining freshness of a food product is provided and may include measuring a first value of one or more electrical properties of one or more sensors in a food sensor device prior to the one or more sensors being exposed to an air flow received from adjacent a food product for testing, exposing the one or more of the sensors to a the air flow and measuring a second value of the one or more electrical properties of the one or more sensors. The method may also include purging the air from around the one or more sensors, measuring a third value of the one or more electrical properties of the one or more sensors and comparing the measured values for each of the one or more sensors to determine the freshness of the food product. The method may further include determining an individual freshness result of each of the one or more sensors based on the comparison, determining an ultimate freshness result of the food product based on the individual freshness results of each of the one or more sensors and presenting a visual and/or audio indication of the freshness of the food product.

In another embodiment of the present invention, a system for determining freshness of a food product is provided and may include measuring means for measuring at least one of a first value of one or more electrical properties of one or more sensors in a food sensor device prior to the one or more sensors being exposed to an air flow received from adjacent a food product for testing; and a second value of the one or more electrical properties of the one or more sensors. The system may also include exposing means for exposing the one or more of the sensors to a the air flow and determining means for determining if the second value is greater, by a predetermined amount, than the first value.

In another embodiment of the present invention, a system for determining freshness of a food product may include measuring means for measuring at least one of: a first value of one or more electrical properties of one or more sensors in a food sensor device prior to the one or more sensors being exposed to an air flow received from adjacent a food product for testing, a second value of the one or more electrical properties of the one or more sensors after exposure of the sensors to an air flow obtained from around a food product for testing and a third value of the one or more electrical properties of the one or more sensors. The system may also include exposing means for exposing the one or more of the sensors to a the air flow, purging means for purging air from around the one or more sensors, comparing means for comparing the measured values for each of the one or more sensors to determine the freshness of the food product and determining means for determining at least one of: an individual freshness result of each of the one or more sensors based on the comparison and an ultimate freshness result of the food product based on the individual freshness results of each of the one or more sensors. The system may also include presenting means for presenting a visual and/or audio indication of the freshness of the food product.

In another embodiment of the present invention, a method for manufacturing a material having variable electrical properties upon exposure to at least one of particles, molecules and biological cells may include dissolving or suspending polyaniline in a solvent, ultrasonically agitating the polyaniline-solvent mixture, combining the ultrasonically agitated polyaniline-solvent mixture with an acid and combining the acid-polyaniline-solvent mixture with carbon.

In another embodiment of the present invention, a sensor material capable of variable electrical properties upon exposure to particles, molecules and/or biological cells contained in an airflow may comprise between about 35-70 weight percent polyaniline, between about 5-25 weight percent carbon and between about 15-86 weight percent carbon.

In yet another embodiment of the present invention, a method of attenuating resistance drift in a conductive polymer includes exposing the conductive polymer to ammonia gas, amine vapors and/or spoiled food vapors.

These and other embodiments, objects and advantages of the present invention will become more clear with reference to the following detailed description and attached figures, a brief description of which follows.

### BRIEF DESCRIPTION OF THE DRAWINGS

FIG. 1 is a perspective view of a sensor device according to some embodiments of the present invention.

FIG. 2A is a cross-sectional view of a sensor device according to some embodiments of the present invention.

FIG. 2B is a perspective exploded schematic of a sensor device according to some embodiments of the present invention.

FIG. 3A is a top view of a motherboard for use in a sensor device according to some embodiments according to the present invention.

FIG. 3B is a top view of a daughterboard for use in a sensor device according to some embodiments according to the present invention.

FIG. 4A is a perspective top view of a button unit according to some embodiments of the present invention.

FIG. 4B is a perspective bottom view of the button unit illustrated in FIG. 4A, according to some embodiments of the present invention.

FIG. 5A is perspective close-up view of a battery compartment for a sensor device according to some embodiments of the present invention.

FIG. 5B is perspective close-up view of a battery compartment door cover for a sensor device according to some embodiments of the present invention.

FIG. 6 is a perspective view of a battery compartment according to some embodiments of the present invention.

FIG. 7 is a perspective close-up view of a portion of a sensor device, illustrating an air-intake portion, according to some embodiments of the present invention.

FIG. 8 is a perspective exploded view of a fan assembly according to some embodiments of the present invention.

FIGS. 8A-8C are views of an alternative design for a fan for the fan assembly according to some embodiments of the present invention.

FIGS. 9A-9C are views of an alternative design for a fan for the fan assembly according to some embodiments of the present invention.

FIG. 10 is a perspective view of an alternative design for a fan for the fan assembly according to some embodiments of the present invention.

FIG. 11 is an exploded perspective view of a sensor card according to some embodiments of the present invention.

FIG. 12 is a front view of a sensor circuit board according to some embodiments of the present invention.

FIG. 13 is a front view of a sensor circuit board according to some embodiments of the present invention.

FIG. 14A is a schematic block diagram of the electrical system according to some of the embodiments of the present invention.

FIG. 14B is a schematic block diagram of the microcontroller fan connection according to some of the embodiments of the present invention.

FIG. 14C illustrates a representation of a control signal for a soft start routine for a sensor device according to some of the embodiments of the present invention.

FIG. 15A is a schematic block diagram of a method for assembling a sensor device according to some embodiments of the present invention.

FIGS. 15B-15K are graphs aiding in the illustrating of algorithms for determining food freshness according to some embodiments of the present invention.

FIGS. 16A-E illustrate a flowchart of the operation of a sensor device according to some embodiments of the present invention.

FIG. 17 illustrates a flowchart for a rolling light process according to some embodiments of the present invention.

FIG. 18 illustrates a voting chart for sensor results for a sensor device according to some embodiments of the present invention.

### DETAILED DESCRIPTION OF THE INVENTION

The features and details of the invention will now be more particularly described. It will be understood that particular embodiments described herein are shown by way of illustration only and do not in any way represent limitations of the invention. The principal features of this invention can be employed in various embodiments without departing from the spirit and scope of the invention. Some embodiments of the present invention may be used in combination or along side of the embodiments disclosed in co-pending and co-owned U.S. published patent application no. 20050153052, entitled “Food and Beverage Quality Sensor,” filed Jan. 13, 2004, the entire disclosure of which is incorporated herein by reference in its entirety.

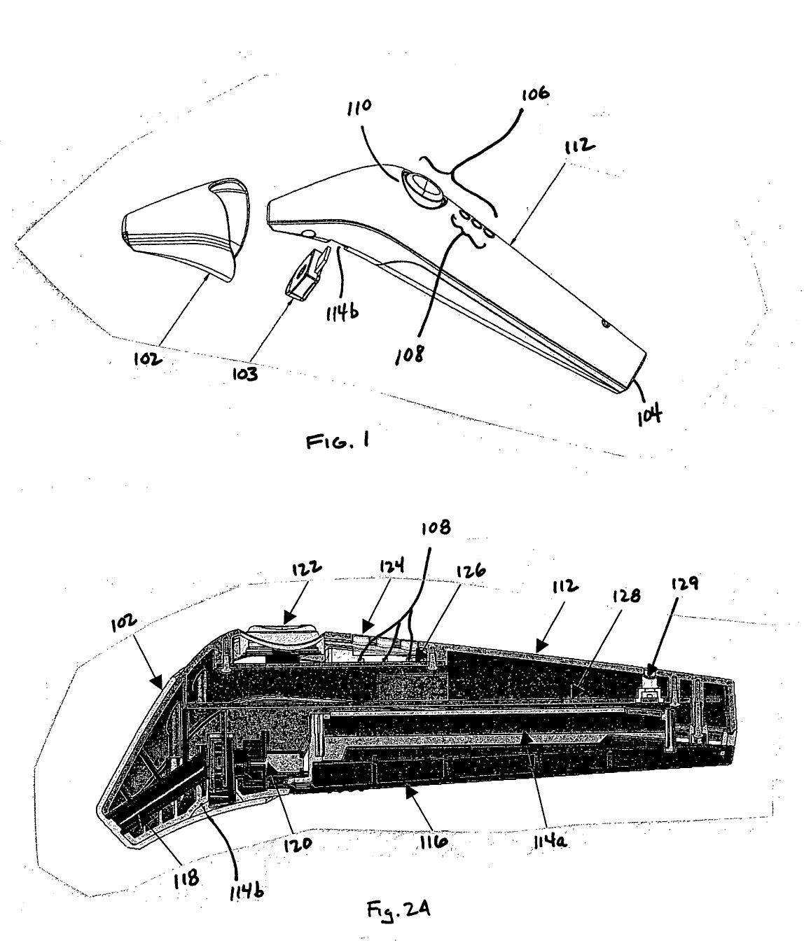

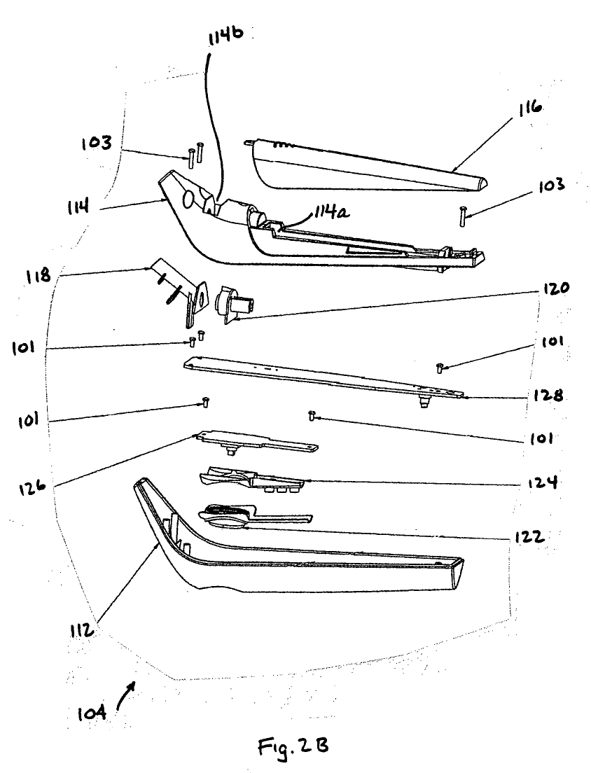

As shown in FIGS. 1, and 2A-2B, one embodiment of the invention is directed to a handheld food sensor device 100 which may include an ergonomic form factor (as illustrated), with a unique head-to-spine angle. The device may also include a nose cover 102 (for use when the sensor device is not in use—protects the internal sensors from any external fumes; e.g., cleaning solutions and the like), a body assembly 104 (for grasping the device) and a sensor card 103 assembly (sensor card).

The sensor device may also include a control portion 106 having indicator lights 108 (e.g., LEDs) the status of which (during operations) may indicate the perishable food state (after analysis is complete), and may also indicate operation status (e.g., on, off, standby, processing). The handheld device may also include a keypad area 110 which may be configured several ways, including a particularly advantageous embodiment featuring a four way switch or a “D-pad” such as is used on PDAs or mobile telephones, or a single, multi-functional switch as shown in FIG. 1 and other figures.

The body subassembly may include a spine 114, which is affixed to back portion 112. Spine 114 may further include a battery compartment 114a and sensor card slot 114b. The subassembly may also include a battery door 116, intake 118 and fan assembly 120. In some embodiments, other parts (with the exception of fasteners 101 and 103), may mount to the back portion: e.g., button/gasket 122, triple lens 124 (covering the LEDs), daughterboard 126 and motherboard 128. A sensor card replacement time indicator light/LED 129 may also be provided in addition to the LEDs 108. It is worth noting that the components of the sensor device may be designed to snap fit together, thus eliminating a substantial portion (or all) screw-fasteners from the design.

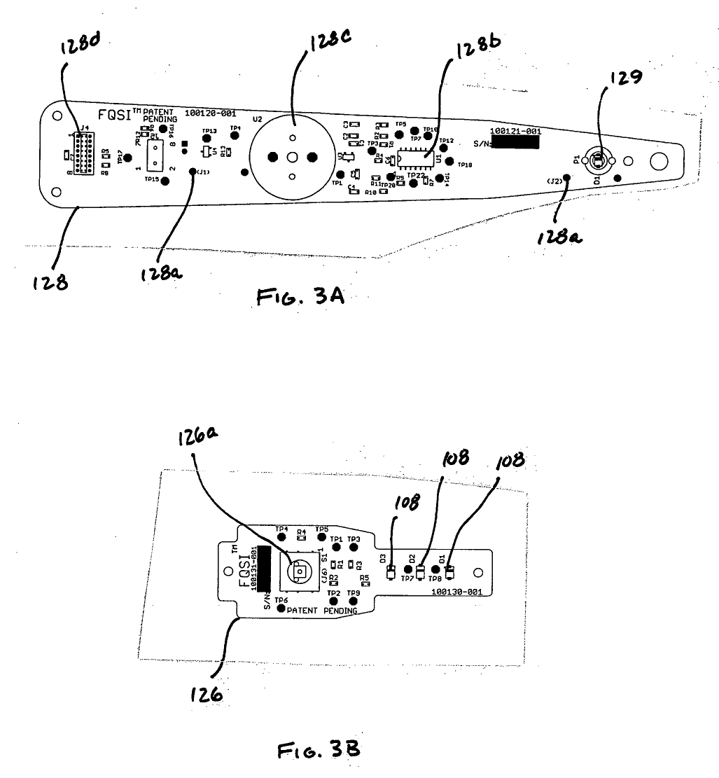

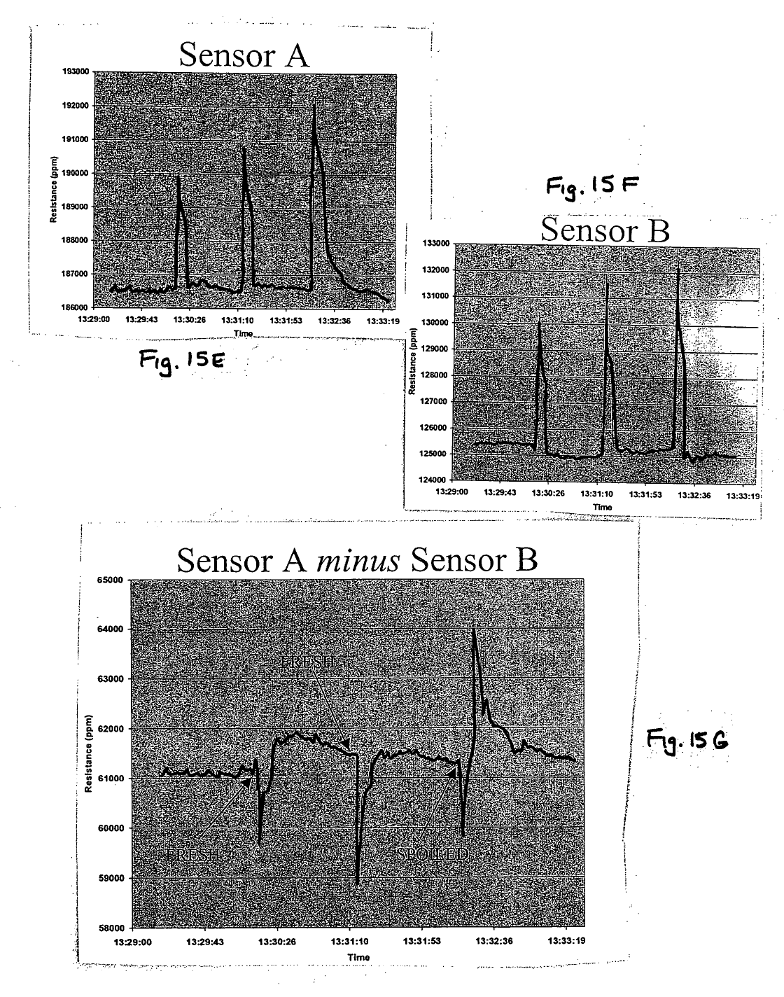

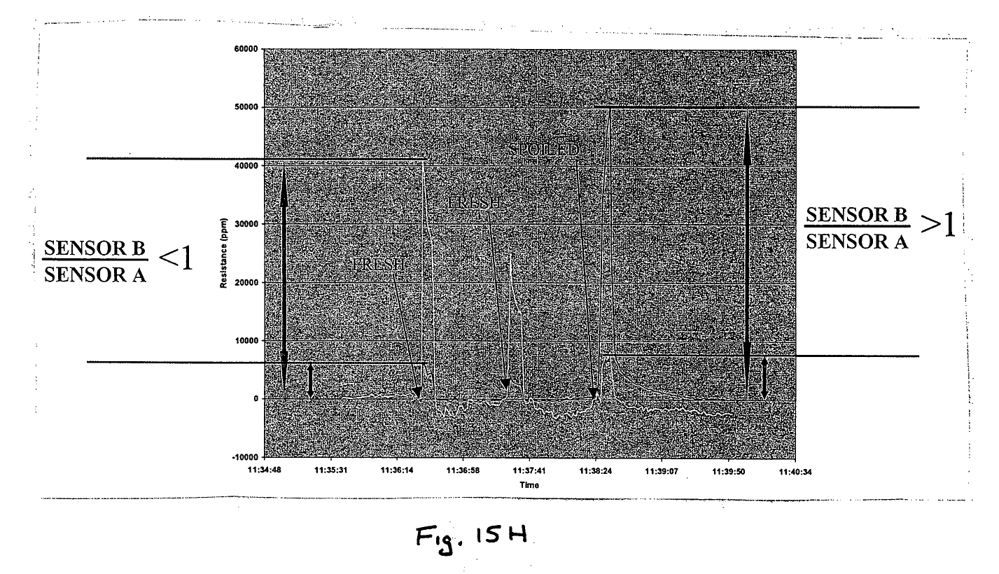

As shown in FIG. 3A, motherboard 128 may include various electronic components which together allow the device to determine the perishability/freshness of a sampled food product (in carrying out some of the embodiments of the present invention), and for carrying out other associated processes (e.g., low battery). The motherboard may include one or more of the following: negative and positive battery contacts 128a, a microcontroller (see FIG. 14A and corresponding written description) 128b, one or more switches, a piezo buzzer 128c (or other sound/speaker device), LED 129, resistors (and other electronic components), capacitors, diodes, transistors, memory and electrical and mechanical connectors (e.g., 128d).

The daughterboard, as shown in FIG. 3B, may include the same or similar components. In some embodiments, the daughterboard may include one or more of the following: negative and positive battery/power contacts, a microcontroller, one or more switches 126a (at least one may correspond to button unit 122), a piezo buzzer (or other sound/speaker device), LED(s) 108, resistors (and other electronic components), capacitors, diodes, transistors, memory and electrical and mechanical connectors.

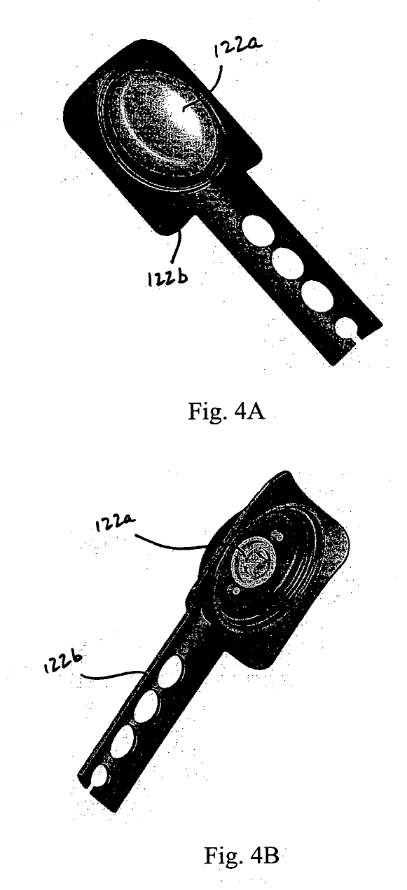

FIGS. 4A and 4B represent button unit 122, which may be used to turn on (awaken) the sensor device, make selections for determining the type of food to be sampled, as well as initiating the food sampling process. The manufacture of button unit 122 ensures a gap-less device. Specifically, button portion 122a may be made via an injection molding process, using, for example, ABS material. Thereafter, the molded button portion 122a may then be inserted into a second molding tool, where a second component—button mount 122b, made of a second material (e.g., thermoplastic elastomer—TPE), is injection molded onto the button portion 122a, forming a plastic-to-plastic bond. This bond produces a gasket like effect eliminating gaps from final product so that the internal electronics are protected from liquids and other foreign matter (e.g., dust/dirt).

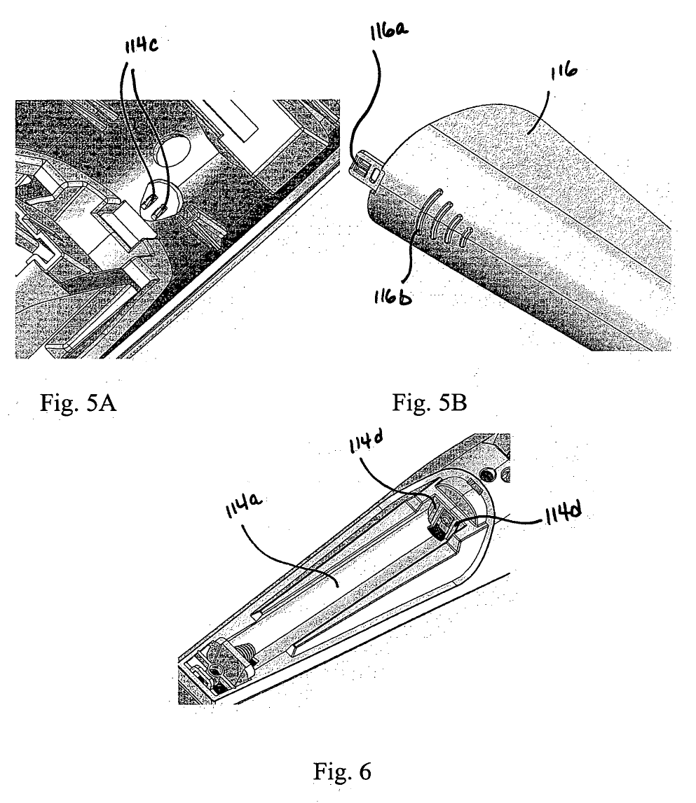

FIGS. 5A-5B illustrate the battery compartment 114a and cover 116. While the battery door may be held in place using any known methods familiar to those of skill in the art (e.g., a screw, snapfit and the like), the battery door according to some embodiments of the present invention may be held in place by using an atypical snapfit feature. Specifically, as shown in the figures, the snap is preferably in the same plane as the sliding motion used to remove the battery door. To remove the door, a user overwhelms the snapfit with enough force so that protrusions 114c, located in the spine part 114, slide out of indentations 116a located in an engagement portion of the battery door 116. The battery door may also include a gripping portion 116b to aid in applying force to remove the door.

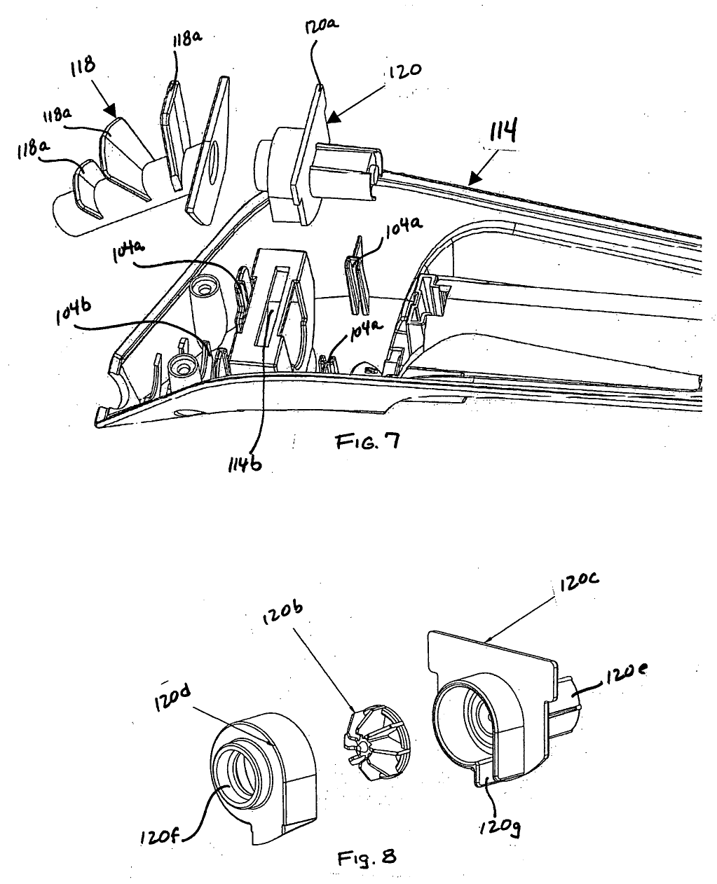

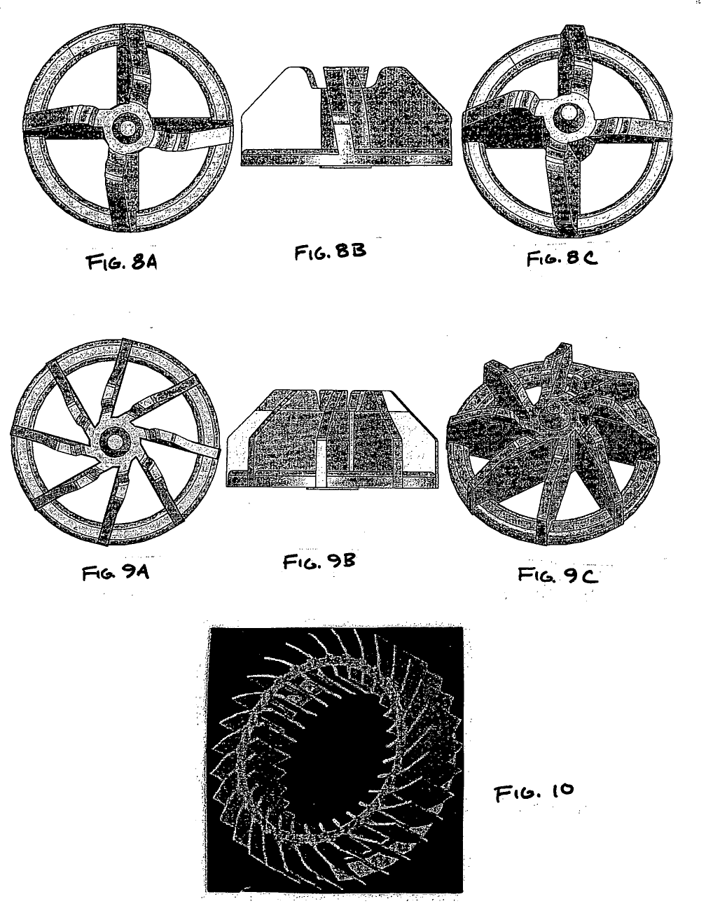

The device may be conveniently powered by either a replaceable or rechargeable power source (e.g., AA batteries), and may include a built-in low-power circuit to notify a user of a low power situation. Such circuits are known in the art and may be used with some embodiments of the present invention. Moreover, as shown in FIG. 6, the device may also include a battery orientation protector to prevent upside down battery insertion. While such protection may be accomplished via an low-power electrical circuit, some embodiments of the present invention make use of a simple and cost effective mechanical feature. As shown, the battery compartment 114a includes protrusions 114d, which only allow a positive terminal of the battery to contact a positive terminal only. Thus, there is no electrical connection upon placement of a negative terminal of a battery adjacent the positive battery terminal contact in the battery compartment.

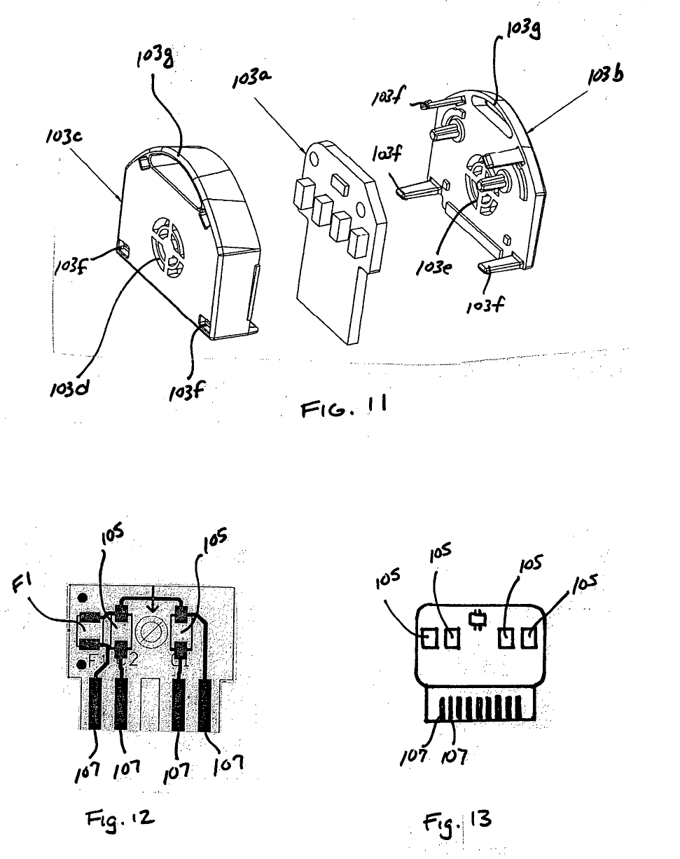

FIG. 7 illustrates a perspective view of an upper end of the spine 114 of the body subassembly 104. As shown, fan assembly 120 may be received in portions 104a of the spine 114, and intake 118 may be received in portions 104b. The intake 118 includes projections 118a and the fan assembly 120 includes portion 120a which conform to portions of the back portion 112 and the mother board 128, respectively, such that these components may be held in place in the finished assembly. It is worth noting that components, such as those listed above, may be integrated into the design of the spine or back of the sensor device, thereby eliminating the need for a separate component.

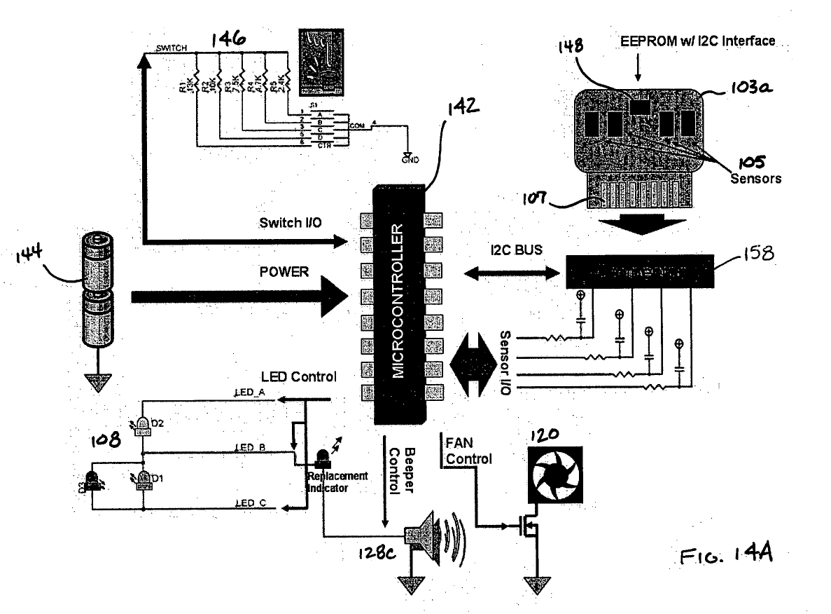

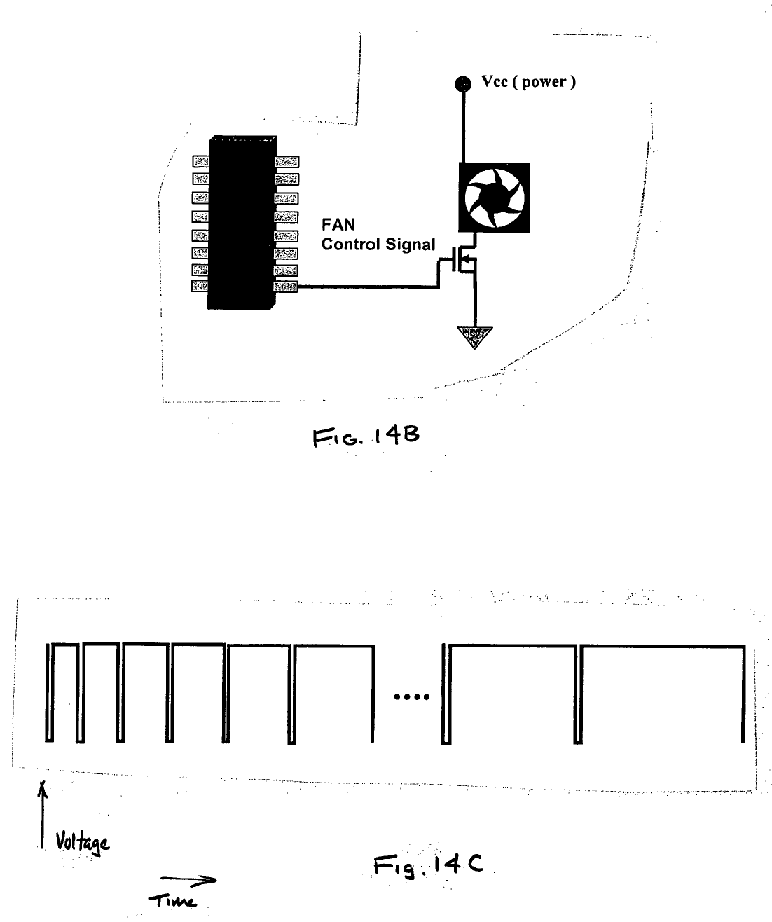

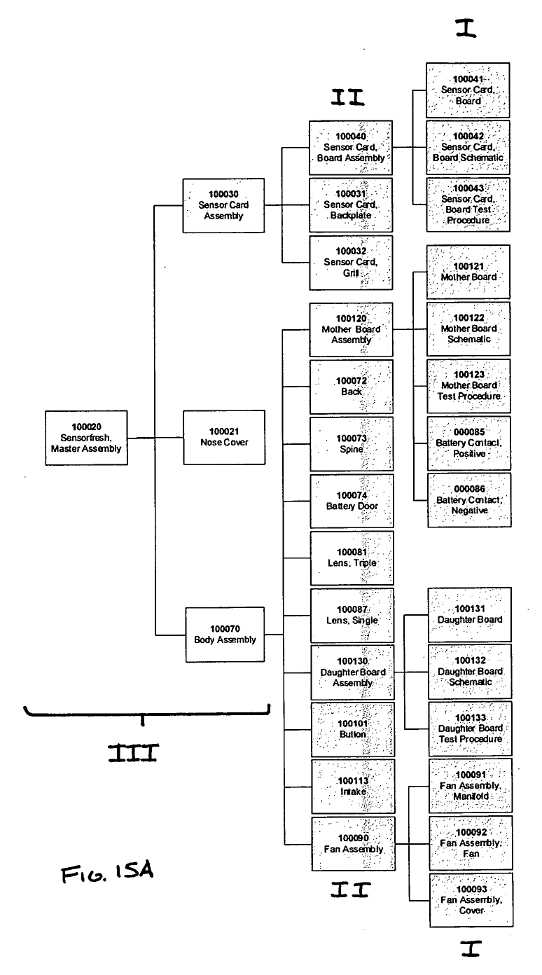

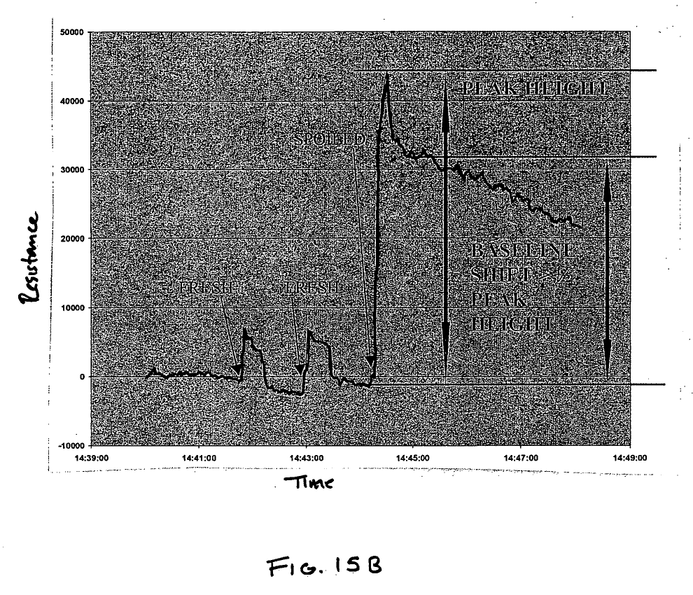

FIG. 8 is a perspective illustration of one embodiment of the fan assembly 120. As shown, the fan assembly may include a fan 120b, a fan manifold 120c, and a fan cover 120d. The fan 120b may include alternate configurations as shown in FIGS. 8A-C and FIGS. 9A-C, including a squirrel cage type fan as shown in FIG. 10. Motor 120e is received in the backside of the fan manifold, and may be affixed therein in any manner familiar to one of ordinary skill in the art (e.g., interlocking members, adhesive, frictional fit, and the like). The fan cover may include an inlet 120f, used to draw air into the center of the fan assembly, and an exhaust port 120g, positioned on a side of the fan manifold 120c to vent air to the environment. The operation of the fan assembly may be similar to that of a turbine, moving volumes of air through a cavity (containing the sensor card) between the fan cover and manifold and out of the exhaust port located on a side of the fan manifold. A raised feature (not shown) may be added around the exhaust port to convey to a user, through tactile feel, when the hand of a user is covering the exhaust port. It is worth noting that other types of air-suction devices may be employed as an alternative (or in addition to for redundancy) to the fan assembly. For example, a piston and/or bladder style pump may be used to draw air into the device and across the sensors.

It is also worth noting that it is preferable that internal parts that come into contact with the air sample are made from an inert material (e.g., polypropylene or a material having similar characteristics to polypropylene). Polypropylene, being an inert material, is less likely to absorb or retain any sample odor (i.e., chemical molecules and/or biological particles/organisms) than other materials suitable for injection molding (e.g., ABS). Accordingly, the intake, the sensor card grill, the sensor card backplate, the fan cover, the fan, and the fan manifold are preferably made of such a material. The remainder of the parts may be made of another material (e.g., ABS), with the exception of the gasket material, which is preferably made of a thermoplastic elastomer, and the lenses which may be made of polycarbonate. ABS is preferable for other parts (or a material with similar characteristics) due to its impact strength. Polycarbonate is preferable for lenses due to its translucency. One of skill in the art may appreciate that some components, for example the intake, may also be manufactured of flexible tubing.

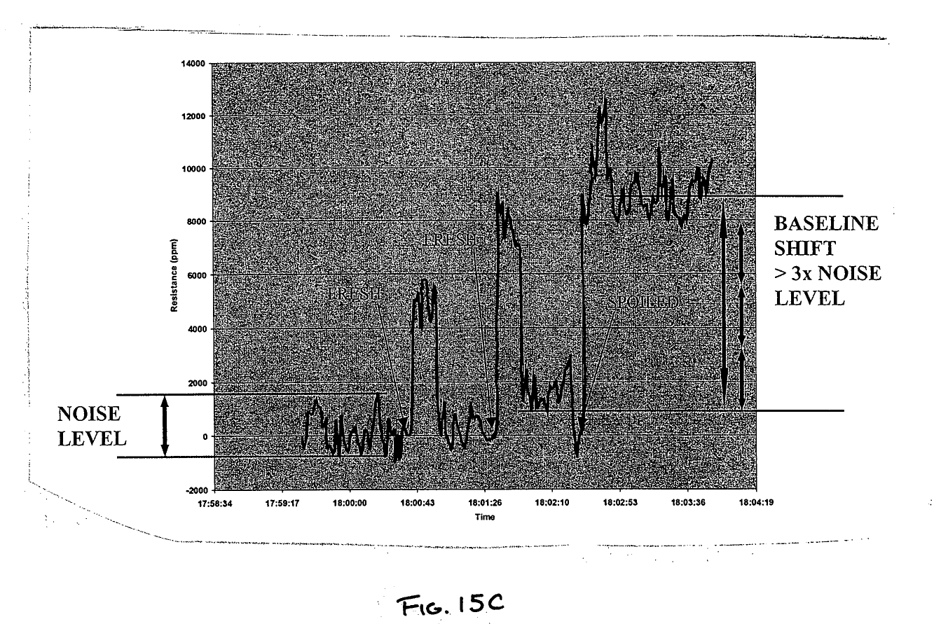

FIG. 11 illustrates the sensor card 103, which may include a sensor circuit board 103a, a sensor backplate 103b and sensor grill 103c. The sensor grill and backplate may be injection molded plastic products, each including an opening(s) 103d, 103e, for airflow. In addition, the sensor grill and backplate may be provided with interlocking members 103f so that the two pieces may be affixed to one another without screw-fasteners, sandwiching in the sensor circuit board therebetween. A gripping portion 103g may also be integrally molded into the grill card and/or backplate portion (or affixed thereto) so that the sensor card may be easily inserted and removed from the sensor card slot 114b on spine 114 of the body subassembly.

The sensor card may also include sterile packaging which is only broken/opened upon use in the device. For example, in one of the embodiments of the invention, the sensor card may include a plastic wrapping which seals both the grid opening 103d and backplate opening 103e of the sensor card. Included within the sensor card slot of the sensor device may be a piercing (e.g., puncture, shredding, opening) device which punctures the wrapping at both 103d and 103e, so that air can flow through these openings. Preferably, the covering to 103d and 103e is completely removed so that it does not trap contaminates. In that regard, such packaging material may be substantially removed by a user.

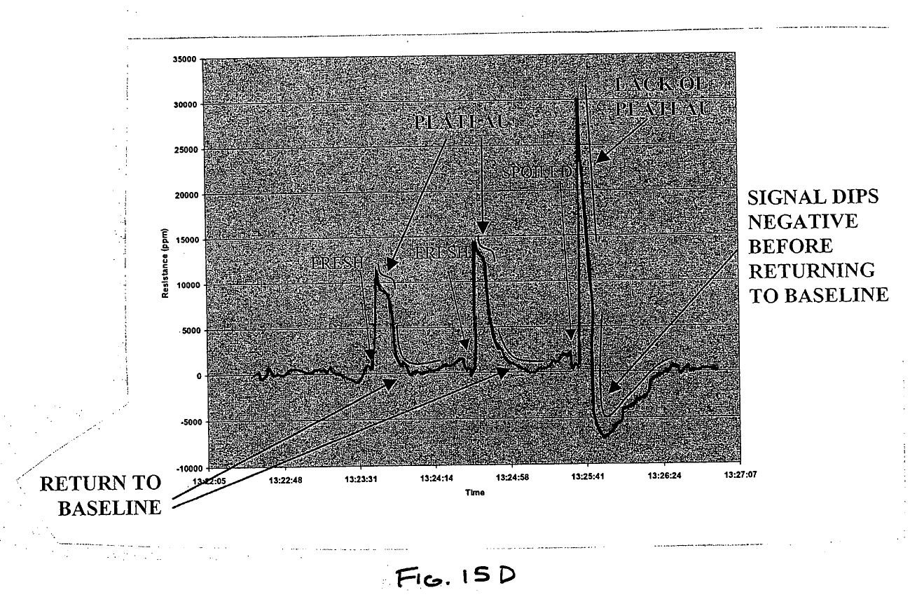

The sensor card is preferably designed to be disposable to ensure high quality analysis. As shown in FIGS. 12-13, the sensor circuit board may comprise single layer (e.g., FR-4) laminate circuit board designed for surface mount electronics (for example), and generally includes one or more sensors 105, which may be connected to contacts 107. The 25 contacts are in turn received into an edge connector in the motherboard (for example). According to one embodiment, as shown in FIG. 12, a fuse (F1) may be included in the sensor circuit board (a two sensor circuit board is illustrated in FIG. 12), to indicate sensor card change: when a number of uses exceeds a predetermined threshold, the fuse blows and the sensor device is inoperative. Alternatively, use of EEPROM memory can be used to determine when a sensor card has expired (see below).

As shown in FIG. 13, preferably, one embodiment of the sensor circuit board includes a side having one or more sensor devices 105 positioned to receive airflow from the intake via the opening in the sensor grill. Air from the intake is directed by the opening 103d in the sensor grill to impinge on one or more sensors 105, flowing thereon and around to exit out the sensor card through opening 103e provided in the backplate. From that point, the air may enter the inlet 120f of the fan cover of the fan assembly, where it is exhausted by the fan 120b out exhaust port 120g.

FIG. 14A represents a schematic circuit diagram of an exemplary electrical system 140 of the sensor device according to some embodiments of the present invention. As shown, a microcontroller 142 having integrated circuitry thereon which receives power from power source 144. Switch(s) 146 provides input to the microcontroller for controlling the status of the operation of the device: e.g., on, off, stand-by, food selection (e.g., beef, chicken, fish, pork) and air-sampling. While in some embodiments of the present invention, a D-switch may be used to select the type of food, where each direction of the switch represents a food type, other embodiments of the invention use a unique freshness algorithm which may be used for any food product (e.g., beef, chicken, fish, pork).

FIG. 14B is a diagram of one embodiment of the invention directed to the connection between the microcontroller and the fan motor. In this embodiment, the fan is preferably connected directly to Vcc (power) which is shared with the microcontroller. When power is applied, before the fan starts to turn, the motor will act as a short, which causes a sudden current sink on power. Since the battery is slow to react to the sudden large demand of the current draw, a resulting voltage drop occurs on the power and is observed on the microcontroller. Accordingly, a soft start routine may be included to the microcontroller code to reduce noise generated by the startup of the fan.

The soft start routine is unlike existing soft start routines commonly use today. Specifically, the common soft start method is a fixed frequency startup with modulating duty cycle. In contrast, the method used in this invention uses a modulating duty cycle and frequency. This is accomplished by keeping the FAN OFF (signal pulse low) for a fixed 2 cycles (i.e., a predetermined number of cycles) while modulating the FAN ON (signal pulse high) time from about 11 cycles to about 32 cycles. The ON time is changed after every 32 periods (or thereabout) in an increment of 6 cycles (for example). This routine reduces the voltage swing be within 0.58 v (for example) and while reducing the voltage settling time to be under 17 ms (for example). FIG. 14C illustrates a fan control signal for the soft start routine.

In still other embodiments, selection of food type may be accomplished by pressing the button unit 122 repeated times to “scroll” through one of the predetermined selections, whereby after each press of the button, either or both of a visual and audio indication is presented to let the user know that a particular selection has been made. Thus, after a single press of the button, the device may flash all the LEDs once and/or give a single “beep” via the piezo buzzer (e.g., selection of chicken), after another button press, all the LEDs may flash twice and the piezo buzzer may beep twice (e.g., selection of beef), and so on (e.g., 3 flashes/beeps for the third selection, four flashes/beeps for the fourth selection). An extended button press (e.g., greater than about one or about two seconds) may activate the testing sequence.

The microcontroller may include a memory (the memory may also be provided separately) to store a control/operation program of the sensor device, or the control/operation program may be “hard-wired” into the circuitry of thereof. The microcontroller and/or the motherboard may include circuitry for measuring one or more electrical properties of one or more sensors in the sensor card (and/or other parameters of the sensor device and sensor card). Alternatively, the sensor device may include a separate memory card port for accepting a memory card and/or updated microprocessor (e.g., compactflash, memory stick, SD memory, smartmedia, and the like), to keep track of card parameters and the like. The sensor device may also include one or more communication ports for communicating information to the microcontroller, the sensor card and/or a memory of the sensor device (e.g., USB, infrared, serial, radio-frequency, parallel, SCSI, firewire, and the like). Accordingly, the microcontroller may control one or more LEDs 108, the audio output device 128c (e.g., speaker, piezo-electric buzzer) and the operation of the fan 120 (and/or other air movement device). The sensor card 103a, via electrical contacts 107, also communicates to the microcontroller, such that the microcontroller receives the output (and may store such output) of one or more sensors 105 provided on the sensor card.

The memory of the microcontroller and/or the sensor card may also provide algorithm information for the device, either for general operation or for a particular food product. Such information may be provided via a EEPROM 148 (for example), or any other type of memory storage. The EEPROM may also be used to log a number of uses, duration of usage and/or other parameters related to the card. Moreover, an 12C or SPI bus (or any other data communications bus) may be included with microcontroller and EEPROM to support communication therebetween.

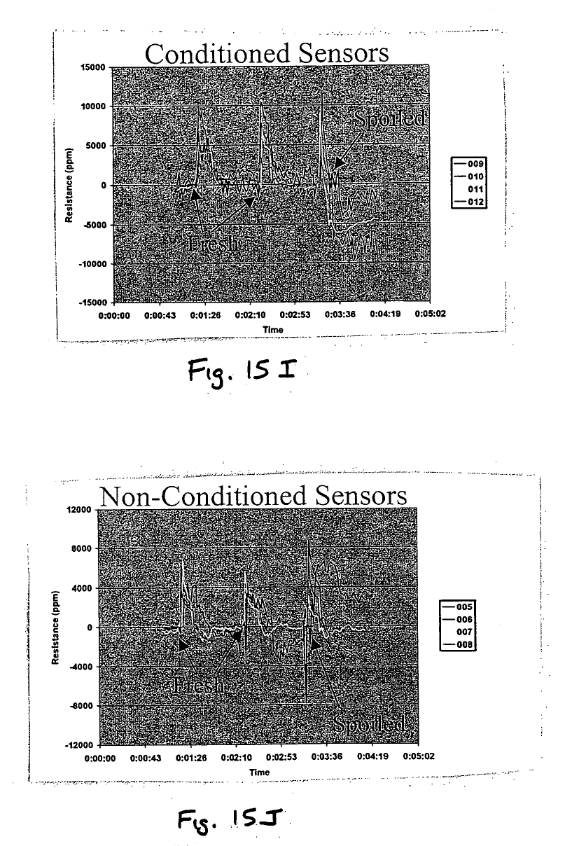

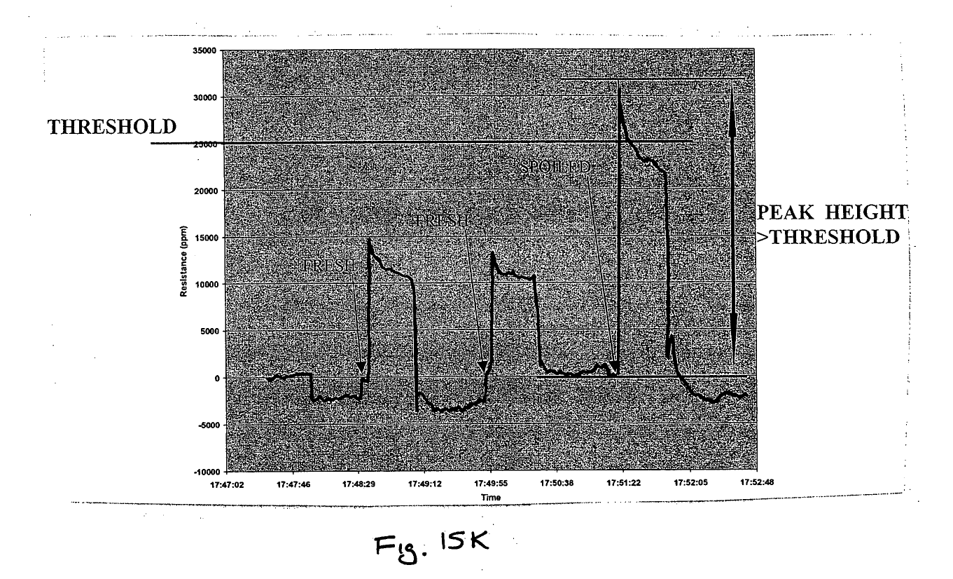

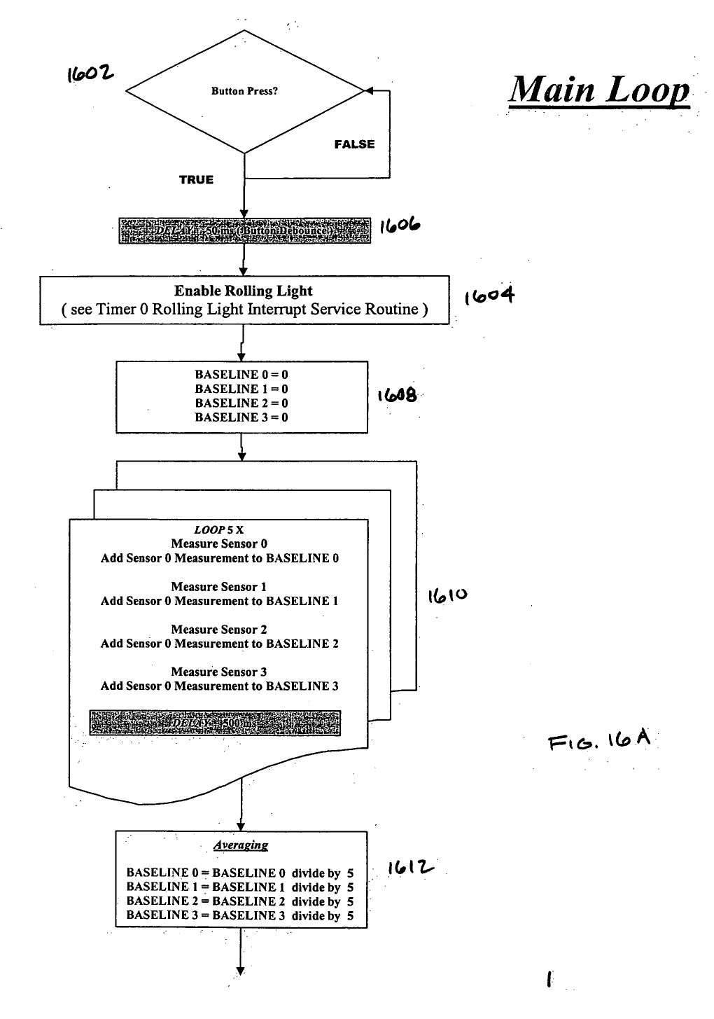

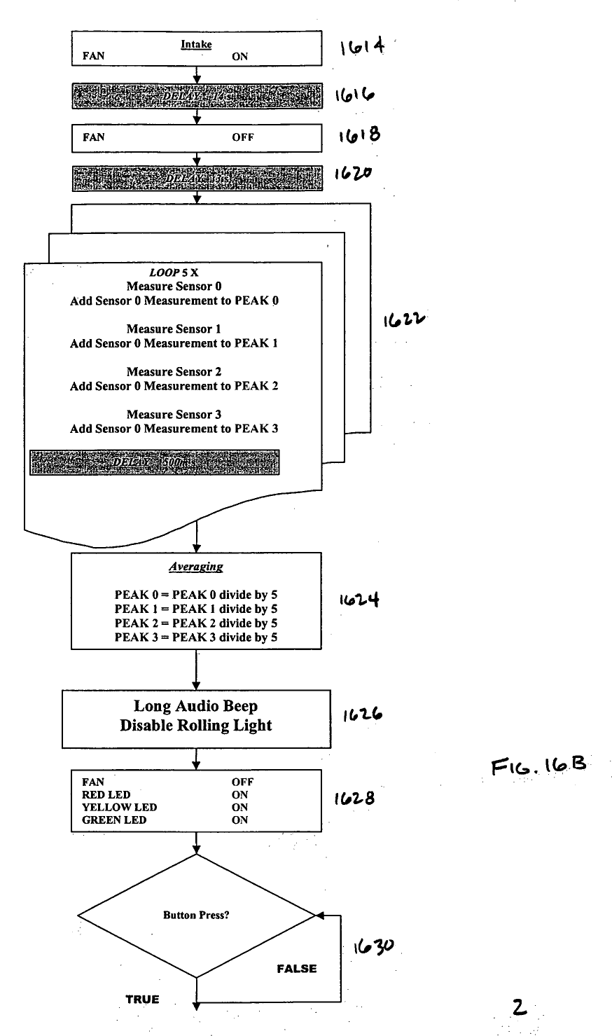

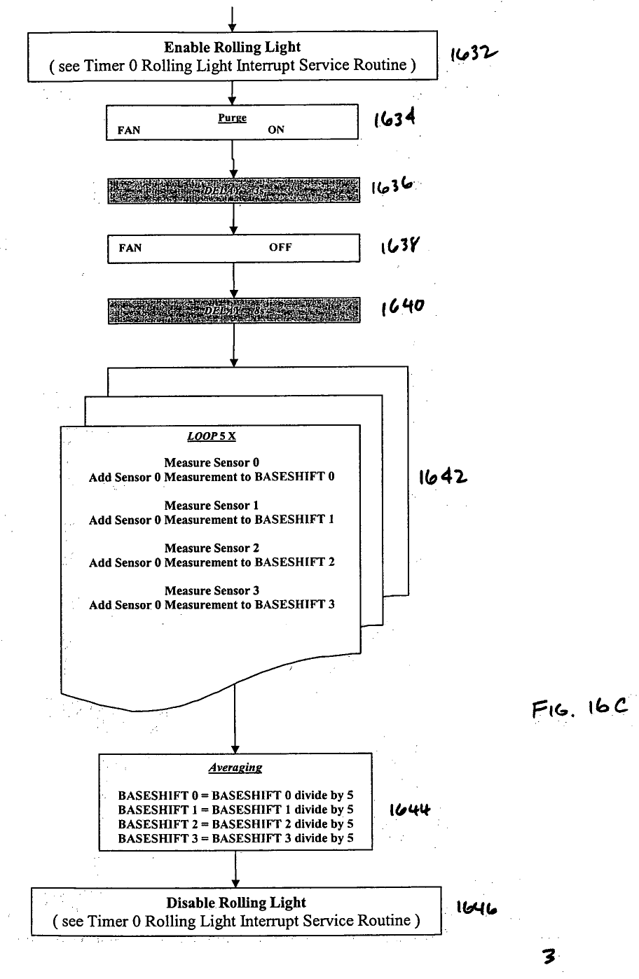

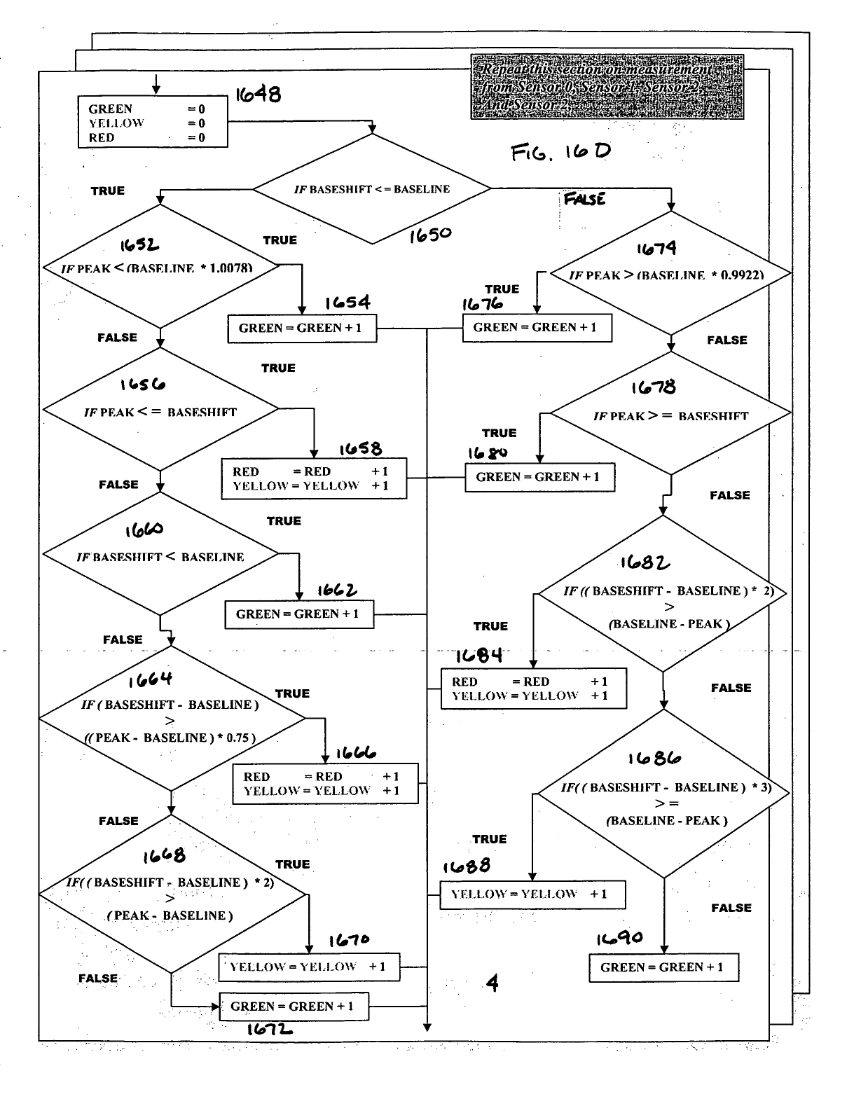

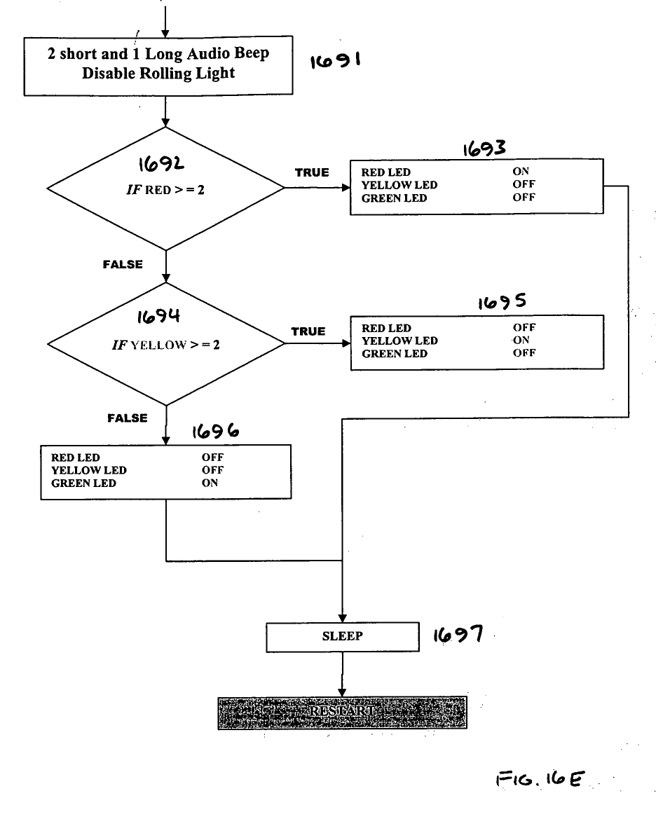

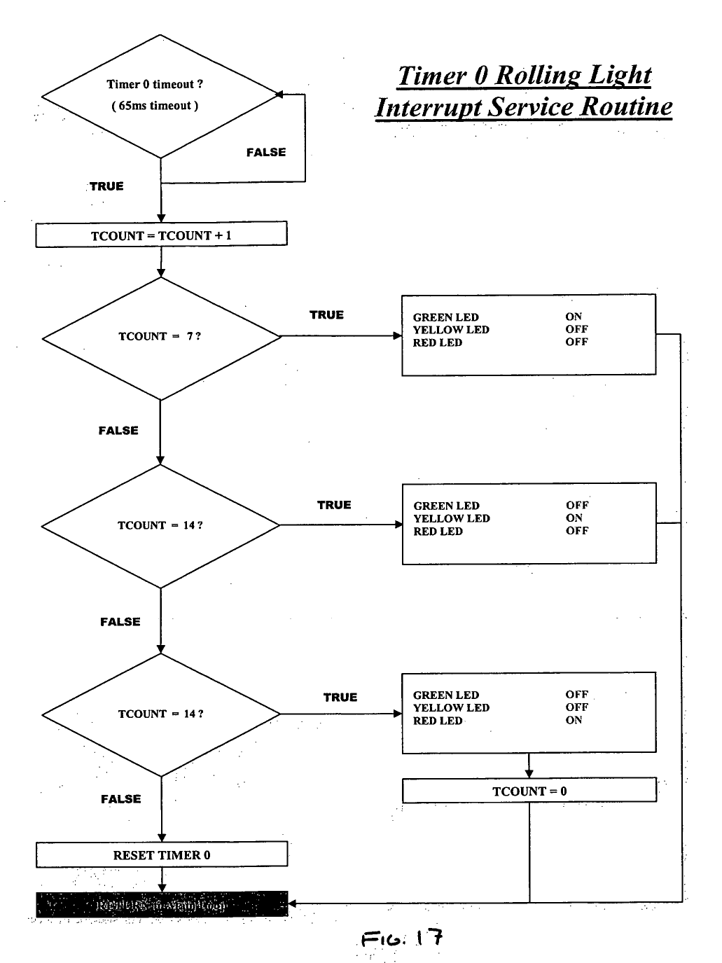

While there may be a multitude of methods for assembling the sensor device in a particular order, FIG. 15 illustrates an overview of one particular assembly order according to one embodiment of the present invention. The chart illustrates three levels of assembly/testing: level I—sub-component level for assembly/testing of the sensor circuit board, assembly/testing of the motherboard and assembly/testing of the daughterboard. Level II—assembly of the main components of the sensor device: assembly of the sensor card and assembly of the of the body subassembly. Level III—assembly of the sensor device: inserting sensor card into the sensor card slot in the body subassembly and nose cover. In that regard, the assembly of the body subassembly may include:
(1) the button/gasket is positioned into the back portion;
(2) the triple lens is positioned on top of the button/gasket;
(3) the daughter board is positioned on top of the triple lens and aligned to the bosses on the back through which the daughter board is screwed down;
(4) the mother board is then positioned into the back and aligned to the bosses on the back through which the mother board is screwed down,
(5) the fan assembly, comprised of the fan, the fan manifold, the fan cover is assembled;
(6) the motor is assembled to the fan manifold,
(7) the fan assembly, with motor attached, is put into place via guide rails in the back;
(8) the intake is put into place via a guide slot in the back;
(9) the spine and its now attached components is attached to the back and its now attached components, with the positive and negative battery spring contacts (not shown) on the mother board guided through their respective openings in the spine;
(10) the spine is screwed to the back;
(11) the batteries are inserted; and
(12) the battery door is slid into place.

With particular regard to assembly of the sensor card, it may be assembled using automated equipment such that a sterile environment may be maintained. The sterile environment is preferred so that the sensors are not contaminated during assembly and prior to use. Using such automated equipment, multiple sensor circuit boards may be produced from a large panel (multiple sensor circuit boards affixed together at the edges). The panel is cleaned using a vapor-phase degreaser (for example). Individual sensor circuit boards may then be snapped apart from the panel.

An Assembly Pallet may be machined for receiving a plurality of sensor backplate components. Cavities may be included with the Assembly Pallet such that components (e.g., backplates) cannot be orientated in the wrong direction. An exemplary Assembly Pallet may be about 8 inches by about 15 inches, which corresponds to a size for loading into a GenRad, Inc. test system for automated circuit testing (i.e., ATE—automated testing equipment). A corresponding Board Pallet may include cavities for receiving the singulated sensor circuit boards, and may also include cavities/members for ensuring the sensor circuit boards are assembled into the Board Pallet in the right direction (e.g., using non-symmetric angled edge feature on the board). The Board Pallet may be as large as any equipment restrictions and reasonable lift weight may allow. The Board Pallet may then be placed onto a Board Pallet Feeder tray.

A Grill Pallet may be loaded with sensor grill parts. As with the other pallets, the Grill Pallet may be machined with particular cavities such that the sensor grill parts cannot be placed in an improper orientation in the Grill Pallet. The Grill Pallet may be as large as the equipment restrictions and reasonable lift weight may allow. A filled Grill Pallet may then be placed onto a Grill Pallet Feeder tray.

Using machine vision, for example, the sensor card may then be assembled by aligning the sensor circuit boards with the sensor backplates. Each sensor grill part from the Grill Pallet may then be snapped into a corresponding backplate and circuit board assembly in the Assembly Pallet, thereby completing the sensor card. Upon completion, the Assembly Pallet is transferred out of the machine. Depending on testing requirements specified, the Assembly Pallet may be loaded into the GenRad, Inc. test station for automated testing or the Assembly Pallet may be placed aside, where an operator can manually (e.g., using an ohm-meter) test the resistance of a pre-defined number of assemblies for quality control.

After the sensor card is assembled, it may be inserted into the body subassembly at slot 114b to establish physical and electrical contact with an edge board connector 158 (see FIG. 14) on the motherboard 128 (see also item 128d on FIG. 3A). In particular, the sensor card is positioned to receive airflow from the intake 118, and exhaust the airflow into the inlet 120f of the fan cover 120d of the fan assembly 120. This enables the sensors within the sensor assembly to be exposed to the airflow. Indentations in the spine may be used (e.g., molded recesses) to facilitate sensor card removal, or may be replaced or augmented by indentations in the spine on both the left and right side of the sensor card. Moreover, a gripping portion 103g (as previously disclosed) may be used to give a good surface for the user to grip when pulling the card out of the device. A tab feature may also be included in the sensor card backplate and grill to facilitate sensor card removal.

In some embodiments of the invention, after the proper placement of the battery into the battery compartment, the device wakes up and performs a system(s) check to determine if the sensor device was awoken from a power down state (no/low batteries in compartment) or from a sleep state. If the device was awoken due to a power-down state, the device will operate a setup procedure, which enables: wake up on interrupt from sleep and the setting of one or more (or all) peripherals to a low power state.

The device may then be placed into a sleep state to minimize the power draw. In the sleep state, the microcontroller monitors the button unit to determine if it has been pressed. Accordingly, when the button is pressed, (and/or other keys in a key/D-pad are moved/pressed, if applicable), the device will wakeup and begin execution (by the microcontroller)of the stored operation program. The program preferably verifies that the current wakeup is due to a key/button press (see above). Once the key press is verified, the device may determine the selected position of the button (e.g., what type of food product is being sampled), and the sampling process is initiated. Each food type may be selected by pressing an individual button corresponding to the type of food (in a multi-button embodiment, or D-pad), or, by pressing the single button unit and scrolling through a list of food categories (illustrated embodiment) where visual and/or audio confirmation of a particular selection may be presented—the selection may be displayed on (for example), and LCD (or LED) display (not shown). Alternatively, no food type selection is necessary in embodiments where the algorithm can determined food product freshness for all food types with the same algorithm.

After air gathered from adjacent the sampled food product has been blown across the sensor(s) in the sensor card, the microcontroller, via one or more algorithms, determines the level of “spoilage” molecules/particles (i.e., freshness) via the change in one or more electrical properties (e.g., resistance, capacitance) of the one or more sensors.

While embodiments of the present invention may operate using only a single sensor in the sensor card, it is preferable that multiple sensors are used. In some embodiments, at least four sensors are used for a three food status (fresh, approaching spoilage, spoiled) system. In such a system, it may be preferable that total sensors not be equal to whole number multipliers of the three food states; e.g., it is preferable to avoid using a number of sensors totally 6, 9, 12, 15 (and so on). However, other embodiments of the invention may include 6, 9, 12 and 15 sensors (i.e., in multiples of three).

A variety of algorithms may be used in conjunction with the resistive change of state sensors according to some of the embodiments of the invention to determine the freshness of sampled food. Such algorithms may include peak height, baseline shift and unique shape algorithms, or a combination thereof. One algorithm may be used with one or all sensors, or separate algorithm types for different sensors. Other algorithms, including (for example) subtraction and relative ratios, typically require an active sensor and a reference sensor. In addition, with multiple sensors, still other algorithms may be used including neural network algorithms and fingerprinting algorithms.

An example of a baseline shift algorithm is illustrated in FIG. 15B. In this example, if the baseline shift of an electrical property of a sensor is greater than one-half the peak value of the electrical property, the tested food product may be considered spoiled. FIG. 15C illustrates another example of a baseline shift algorithm. In this example, baseline shift may also be measured in terms of shift to noise ratio. In the illustrated embodiments, if the baseline shift of the electrical property is greater than three times the noise level, the tested food product is spoiled.

FIG. 15D illustrates an example of a unique shape algorithm. Sensors may exhibit a unique shape—such as the absence of a plateau and/or an overshot of a baseline value of an electrical property. FIGS. 15E-G illustrate three graphs relating to a subtraction algorithm, where an electrical property of two sensors area measured, then the results from one sensor are subtracted from the other sensor. FIG. 15H is a graph of the results of a relative-ratio algorithm. Here, the ratio of peak values of an electrical property are compared to a predetermined set ratio (e.g., 1). In such an algorithm, if the ratio is greater than 1, it may indicate that the tested food product is spoiled.

FIGS. 15I-J are charts representing a conditioning process. In such a process, sensors are exposed to spoilage particles/molecules/cells during fabrication of the sensors (e.g., before the sensor material dries on the sensors). As shown, conditioned sensors shown in FIG. 15I can distinguish fresh from spoiled chicken (as opposed to the sensor results illustrated in FIG. 15J).

FIG. 15K is a chart illustrating the use of a peak height algorithm. In such an algorithm, if the maximum value of an electrical property found during testing is greater than a threshold, the tested food product is spoiled.

In a multi-sensor arrangement, baseline values, peak values and baseshift values of one or more electrical properties for a sensor may be collected and compared to determine food freshness. Accordingly, an ultimate end result indicated by the sensor device depends on the number of sensors which return a particular status/state (i.e., freshness) of the sampled food. The results of each sensor may represent a “voting system” which may be percentage based. For example, if more than 50% of the sensors determine one particular condition (spoiled, approaching spoiled or fresh), then that condition may be the end result/finding of the test.

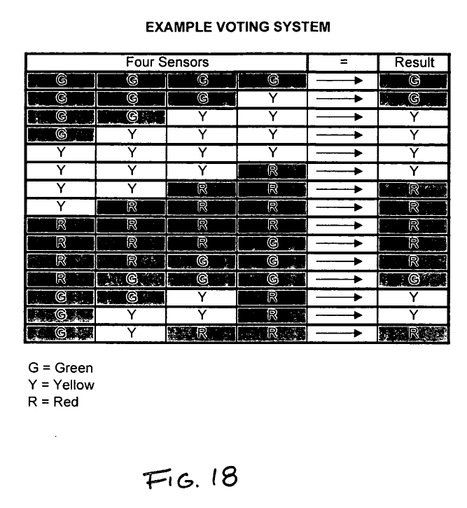

In one particular embodiment of the invention, one algorithm is used to determine food freshness for multiple food types (e.g., one algorithm for beef, chicken, fish, pork) using Baseline, Peak and Baseshift values for one or more electrical properties of a sensor(s). Such electrical properties include at least one of (for example): resistance and capacitance. Accordingly, the operational control program for this embodiment of the sensor device operates according to the flowchart as set out in FIGS. 16A-E. Button unit 122 is pressed to “wake-up” the sensor device to begin a food freshness test. The button press is sensed (1602) by the control program, which enables a rolling/sequential light sequence (1604) to initiate (see FIG. 17). During the rolling light sequence, a Baseline value for one or more electrical properties for each sensor (four sensors in the illustrated embodiment) are set to zero (0) (1608); a delay 1606 may also occur. The current baseline values for the sensors are obtained (1610). As shown, although a single value may be obtained, it is preferable that multiple measurements are taken for each sensor (e.g., 5), and then averaged (1612); prior to performing averaging, a delay may occur (e.g., 500 ms). These values may then stored in a temporary or permanent memory (e.g., memory of the motherboard, daughterboard and/or sensor card).

The fan motor may then be switched on (1614) to obtain a sample of air for analysis by the sensor card. The nose of the sensor device is held above a portion at the food. Preferably, the inlet of the intake at the nose section of the sensor device is positioned between about 0.125 inches and about six (6) inches from the surface of the food, and more preferably between about 0.125 inches and three (3) inches, and most preferably between about 0.125 inches and about one 0.50 inches.

The fan is switched on for a predetermined period of time to insure that the sensors are sufficiently exposed to the airflow (1616). This time period may be between 2-30 seconds, more preferably between about 5-20 seconds, and most preferably about 14 seconds. Thereafter the fan is turned off (1618). The Peak values for the one or more electrical properties of each sensor are then obtained (1622), but preferably the analysis only occurs after a delay (1620) from the point at which the fan is switched off. Similar to the base values, multiple measurements (e.g., 5) of the peak values of each sensor are obtained. Thereafter, the average peak value(s) of the corresponding electrical property is obtained for each sensor (1624) (a slight delay may occur prior to averaging; e.g., 500 ms), which may then be stored in memory (temporary or permanent). The rolling light sequence is then disabled (1626) and a long audio beep may be sounded.

The electrical property may comprise any measurable electrical property of the either the sensor itself, or the sensor in combination with another electrical component. For example, the resistance and capacitance of the sensor may be determined to determine the Baseline, Peak and Baseshift values.

For example, on the motherboard (or daughterboard) there exists a measurement capacitor for each sensor on the sensor card. The measurement capacitor may be selected based upon low temperature coefficient (COG or NPO ceramic type ). The capacitor is first charged up by the controller with an output pin. Once fully charged to a power level, the output pin is switched to a high-impedence mode as an input. The pin than monitors the voltage charge on the cap, which is connected to the sensor on the sensor card. The sensor acts as a discharge path to ground for the measurement capacitor. Since the electrical characteristics (e.g., resistance and capacitance) of the sensor determine current flow, it therefore affects the total discharge time of the measurement capacitor. This discharge time may then be accurately monitored as the microcontroller monitors the voltage on the capacitor pin (e.g., using a 16 bit timer). Such a method may also be used in analog to digital conversion.

The fan is now off and preferably all the LEDs 108 are lit (1628). The user withdraws the sensor device from the food product being tested, and presses the button again (1630) to enable the rolling light sequence (1632) and purge the air from within the sensor card. Air is purged by switching on the fan motor for a period of time (between about 1 s and about 5 s, and preferably about 3 s) (1634, 1636). Thereafter, the fan is switched off (1638). A delay (1640) occurs (between about 2 s and about 10 s, preferably about 8 s), where a Baseshift value for the one or more electrical values are obtained for each sensor (1642), then averaged (1644), which may then also be stored in memory (temporary or permanent). The rolling light sequence is then disabled (1646).

The freshness of the tested food product is then determined by comparing the Baseline, Peak and Baseshift value of one or more electrical properties (e.g., resistance and/or capacitance), for each sensor, as shown in FIG. 16D. A counter for each of the results for each sensor for each result (i.e., RED—spoiled, YELLOW—still fresh but approaching spoiled, and GREEN—fresh) is initialized at zero (0). Step 1648 is done initially only once for a test, then, for each sensor, the steps of the flowchart between 1650 and 1690 may be conducted (depending upon the intermediary results). In other words, steps 1692-1699 are not done until steps 1650-1690 have been evaluated for substantially all (preferably all) the sensors.

Accordingly, for each sensor: a determination is made as to whether the Baseshift value is less than or equal to the baseline value (1650). If true, then a determination (1652) is made as to whether the Peak value is less than the Baseline value multiplied by a constant (in one embodiment, this constant being about 1.0078). If true, then the GREEN counter is advanced by one (1) (1654). If false, then a determination is made as to whether the Peak value is less than or equal to the Baseshift value (1656). If true, the both the RED and YELLOW counters are advanced by one (1) (1658). If false, a determination is then made as to whether the BaseShift value is less than the Baseline value (1660). If true, the GREEN counter is increased by one (1662). If false, then a determination is made as to whether the resultant value of the Baseline value subtracted from the Baseshift value is greater than the resultant value of the Baseline value subtracted from the Peak value, that total multiplied by a constant (in this embodiment, about 0.75) (1664). If true, both the RED and YELLOW counters are increased by one (1666). If false, a determination is made as to whether the resultant value of a constant (in this embodiment, about 2) multiplied by the result of the Baseline value subtracted from the Baseshift value, is greater than the resultant value of the Baseline value subtracted from the Peak value (1668). If true, the YELLOW counter is advanced by one. If false, the GREEN counter is advanced by one.

If the result determined in (1650) is false, a determination is made as to whether the Peak value is greater than the Baseline value multiplied by another constant (in this embodiment, about 0.9922) (1674). If true, the GREEN counter is advanced by one (1) (1676). If false, it is then determined whether the Peak Value is greater than or equal to the Baseshift value (1678). If true, the GREEN counter is advanced by one (1680). If false, a determination is then made as to whether the result of a constant (in this embodiment, about 2) being multiplied by a resultant value of the Baseline value subtracted from the Baseshift value is greater than the resultant value of the Peak value being subtracted from the Baseline value (1682). If true, then both the RED and YELLOW counters are advanced by one (1684). If false, a determination is then made as to whether the result of a constant (in this embodiment, about 3) multiplied by the result of the Baseline value being subtracted from Baseshift value is greater than or equal to the resultant value of the Peak value subtracted from the Baseline value (1686). If true, then the YELLOW counter is advanced by one (1688). If false, the GREEN counter is advanced by one (1690).

As stated earlier, the above process (from step 1650-1690) is conducted for each of the sensors. After the results have been determined for all the sensors, the sensor devices sounds off two short and one long audio beep; or some other arrangement of beeps/buzzes, LED lighting, and the like (1691). Accordingly, in a four sensor system, if the RED counter is greater than or equal to two (2) (1692), then the Red LED light 108 is lit (1693); food product is spoiled. If the RED counter is less than two (2), then a determination is made as to whether the YELLOW counter is greater than or equal to two (2) (1694). If true, then the Yellow LED light 108 is lit (1695); food is till fresh, but approaching spoilage. If the YELLOW counter is less than two (2), then the green LED is lit (1696); food is fresh. After one of the LED is lit indicating the test result (preferably between about 2 s and about 20 s), the sensor device goes into a sleep mode (1697), and the process may be restarted (i.e., return to FIG. 16A to test a new food product, or re-conduct the test on the same food product).

FIG. 18 is a chart illustrating possible results for a four (4) sensor system described above. As shown, a G vote (green) indicates a result that the sampled food is fresh, a Y vote (yellow) indicates a result that the sampled food is approaching spoilage, and an R vote (red) indicates a result that the sampled food is spoiled (votes are tracked by counters for each state as discussed above). Thus, according to this embodiment, if three or more sensors indicate a particular result (i.e., a majority), then the this result is ultimately displayed by the food sensor device by visual and/or audible means (e.g., lighting of a green LED for fresh, yellow LED for approaching spoilage, and a red LED for spoiled). If there is an equal split between two freshness states, the lesser food quality state is chosen. For example, if two sensors indicate the food is spoiled, and two sensors indicate the food is fresh, then the system presents the spoiled LED/sound.

Thus, in the four sensor, three-freshness status result system, there will always be at least two sensors indicating the same result. As the chart illustrates, out of the 15 possible sensor results, six (6) will end in an ultimate result that the sampled food is spoiled, six (6) will end in an ultimate result that the sampled food is approaching spoilage, and only three (3) will end in an ultimate result that the sampled food is fresh. Other embodiments may include similar voting schemes for greater than four (4) sensors.

FIG. 17 illustrates an example of the rolling LED light sequence. As illustrated, a timer is initiated, in which a specific color LED light is switched on for a short period of time (between about 0.2 s to about 1 s), and then switched off, then a second color LED light is switched on/off, and so on. Since (in one embodiment) the LEDs are physically positioned one after another, a “rolling” lighting effect occurs. This may occur for a predetermined period of time (e.g., between about 5 second and 20 seconds), and is generally setup to continue until a result of freshness is determined by the microprocessor.

In another embodiment, the following algorithm may be used in either a single or multiple sensor arrangement (multiple sensors may include a voting system as in a previous described embodiment). Accordingly, for each sensor (or for the sensor), if the Peak Value of the electrical property (properties) is greater than about 103% of the Baseline Value, then the sampled food is considered spoiled. This threshold may be defined as a bacterial level of about 10 million colony-forming units per gram (cfu/g). If the Peak Value of the electrical property (properties) is less than 101% of the Baseline Value, then the sampled food is considered fresh. If the Peak Value of the electrical property (properties) is greater than or equal to 101% of Baseline Value and less than or equal to 103% of Baseline Value, then the sampled food is considered still good for consumption, but approaching spoilage. Accordingly, the result may be displayed via the LEDs (i.e. green—fresh, yellow—approaching spoilage, and red—spoiled.

The sensor device may be advantageously designed to ignore errant input from the user, e.g., if the user presses the selector button at any time after the analysis is begun (e.g., after food selection), the action will be ignored by the circuit. If the user does not release pressure on the button in the food selection step, the remaining steps may be executed as if the button had been released. However, in some embodiments of the invention, in order for the user to take another measurement (which preferably is not done until after the analysis cycle is completed), the user releases the button first before pressing it again.

In some embodiments of the invention, if the button is inadvertently pressed down while in storage, and remains pressed, the cycle will execute only once and the power will then shut off. The device may also be designed to not operate without a sensor card, or not operate with an expired sensor card inserted into the device. In that regard, LED 129 may light to notify the user to replace the sensor card when an alert condition is established, e.g., when the resistance measured by the internal circuitry is above a certain value (i.e., the sensor card is expired). In yet another embodiment, the sensor heads may be serialized by incorporating known resistors.

In some embodiments of the present invention, the sensors used to determined food freshness may comprise a detection material with a resistive (or other electrical) property. Specifically, upon being exposed to an airflow having particles/molecules indicative of food spoilage, an electrical property of the sensor changes (e.g., decreases/increases relative to a baseline). Such sensors may comprise available, commodity items (e.g., a ceramic chip capacitors) which are known in the art. For example, the detection material may be an imprinted polymer or an organic coating including a conductive material (e.g., carbon black polymer resistors, polymer imprinted with carbon black). See Lonergran et al., “Array-Based Vapor Sensing Using Chemically Sensitive, Carbon Black-Polymer Resistors,” Chemistry of Materials 8:2298-2312 (1996), the entire disclosure of which is herein incorporated by reference.

While known resistive varying sensors may be used, some embodiments of the present invention are directed to a sensor having an improved resistive varying material for use in, for example, ceramic capacitors, as well as a new process for making such a material and for making sensors therefrom. Accordingly, the new sensors may be formed from a new detection material which is deposited on a substrate, with an electrical lead provided on both sides of the substrate.

In one embodiment, a material capable of variable electrical properties may comprise between about 35-70 weight percent polyaniline, between about 5-25 weight percent carbon, and between about 15-86% sulfuric acid (or comparable acid). More preferably, the material may comprise between about 45-60 weight percent polyaniline, between about 10-20 weight percent carbon, and between about 25-60% sulfuric acid. Most preferably, the material comprises about 56 weight percent polyaniline, about 14 weight percent carbon, and about 30% sulfuric acid. In addition to these amounts, the components are preferably combined with a solvent, to more easily combine the components and apply the mixture to the ceramic substrate. To that end, approximately 100-500 parts of solvent (e.g., tetrahydrofuran (THF), isopropanol (IPA), hexafluoro isopropanol (HFIP), IPA/water systems, and the like) may be used, more preferably about 100-300 parts solvent, and most preferably about 200 parts solvent, yielding a solution of the material. In some embodiments, particle size control is desirable and polymeric binders known in the art may also be employed. Preferably, particle size should be less than 100 nanometers.

In one embodiment of the invention, the polyaniline is first dissolved or suspended in the solvent, and subjected to ultrasonic waves for a predetermined period of time: between about 5 min and about 30 min, preferably between about 10 min and about 20 min, and most preferably about 15 min. The acid may then be added to the solution and mixed together. Finally, the carbon may be added and the resultant mixture may (preferably) be subjected to additional ultrasonic mixing for a predetermined period of time: between about 5 min and about 30 min, more preferably between about 10 min and about 20 min, and most preferably about 15 min. Preferably, prior to each use (i.e., application to a ceramic), the mixture is subjected to the ultrasonic mixing for the same or similar time periods. Standard laboratory and/or industrial mixing, transfer, ultrasonic and containment equipment may be used manufacture the material.

Immediately after ultrasonic mixing, the mixture may be deposited upon the ceramic substrate/capacitor. Preferably, immediately prior to application of the mixture, the ceramic substrate is cleaned thoroughly by vapor phase degreasing (for example), although other cleaning methods may be used such as plasma cleaning, manually wiping the surfaces with isopropyl alcohol, and the like.

The volume of mixture dispensed onto the ceramic may be between about 0.25 microliter and 2 microliters, more preferably between about 0.50 microliter and 1.50 microliter, and most preferably about 1 microliter. In some embodiments, the amount of mixture applied is of particular importance, as the amount of the material capable of variable electrical properties typically determines the initial resistive value of the sensor and how easily the resulting sensor can determine a change in resistance/capacitance (i.e., one or more electrical properties).

The mixture may be deposited by spraying, e.g., as an aerosol, onto the ceramic substrate, such that the material overlays the electrical leads. Alternatively, the mixture may be poured onto an array of substrates or onto a large substrate (having corresponding electrical leads), and then divided into individual sensor substrates, either manually or via a machine. The material may also be applied to the substrate by coating, dipping, submersion, stamping or electrostatic deposition of the substrate. The solution may also be applied using a manual or automated micropipette method for applying controlled volumes of the solution onto the substrate, e.g., applying a drop, allowing the solvent to evaporate, then applying additional sensor solution (if necessary).

In some embodiments of the present invention, sensors are conditioned to attenuate resistance drift. Normally, with age, the resistance of sensors increases—this condition is known as “drift”. After a certain period of time, the resistance reaches a point which is not usable for embodiments of the present invention. Accordingly, the Applicants have found that by subjecting the sensors (either before or after drying) to at least one of ammonia gas, amines (e.g., derivatives of ammonia) and/or spoiled food vapors, this sensor drift condition may be attenuated. Specifically, after exposing the sensors over a predetermined period of time to ammonia gas, the resistance of the sensor is elevated. The resistance then decreases over a period of time. This decrease counteracts the drift of the resistance of the sensor upward. Thus, storage/shelf life of the sensors may be increased, and in some embodiments, dramatically increased.

Thus, preferably after a predetermined period of drying time, the new sensors may be exposed to ammonia gas for between about several seconds to about a minute, and more preferably between about several seconds and 30 seconds, and most preferably about 10 seconds. The predetermined period of drying time is preferably between about 1 hour to about 10 hours, more preferably about 3 hours to about 8 hours, and most preferably about 7 hours.

After being applied to the substrate, the solvent evaporates leaving a thin film of the conductor-detection material across the electrical leads. A polymer coating may also be applied to the sensor material after the solvent has evaporated, being applied in the same or similar manner as the conductive material. In one embodiment, the polymer coating may be mixed with the new conductive material and applied therewith (i.e., a polymer/solvent solution). One of skill in the art will appreciate that other materials, including gold, silver, and copper, may also be used with embodiments of the conductor/sensor material to yield different initial and test-sample electrical property values. For example, typically, using the material sensors according to embodiments of the new invention, the thin film has a resistance of between about 1 kΩ and about 1000 kΩ prior to exposure to the contaminant, more preferably between about 50 kΩ and about 500 kΩ, and most preferably between about 50 kΩ and about 100 kΩ.

In some embodiments of the invention, the dimensions of the sensor substrate may also play a role in the initial resistance value of the sensor. For example, sensors with the same width and length have the same starting resistance for a given thickness. In that regard, sensors with small width/length ratios typically include a short range of resolution before becoming an open circuit. However, large width/length ratios have poor resolution. Thus, it is preferable, in some embodiments of the present invention to have a width/length ratio close to one (1).

Moreover, the area of width multiplied by length may also play a role for how much sensor material is required to achieve a given resistance. Smaller substrates generally lead to a greater analyte-to-sensor ratio and, therefore, greater sensitivity. Accordingly, using an SMT capacitor as a substrate is preferable, since such substrates may be mass produced easily and inexpensively, available in a width/length ratio of between about 0.75 and about 1.25, and preferably about 1 (e.g., about 0.875), and includes a small area for good resolution. Furthermore, the typically small amount of capacitance may aid in filtering noise from the sensor signal.

The device of the present invention may be used in industrial and military venues, and can be adapted to also incorporate temperature sensors and algorithms to predict spoilage timelines based on temperature histories, thus allowing for further precision.

Other embodiments of the sensor device may include: ruggedized housing to withstand drop and other impact; waterproof or water resistant housing; clock functionality and/or alarm functionality; digital readout/display; humidity sensor; temperature sensor (provided on one or more of the following—the sensor circuit board, the motherboard, and daughterboard)—this may include an IR temperature measurement capability with or without a laser guide. NPO-COG capacitors may be used, which achieve a lower temperature coefficient helping to reduce measurement variation due to temperature change. In addition, one or more additional series resistors may be used for the signal path between sensor and I/O pin to reduce surge current from ESD/EMI. Internal pull-up resistors may enable the reduction of the need for external pull-up resistors (the pull-up helps eliminate the float pin—the float input pin may result in higher power consumption) when the I/O is set to input state during sleep. Also, a test pad may be included to the mother and daughter board for in-circuit testing and in-circuit programming.

Those skilled in the art will recognize, or be able to ascertain using no more than routine experimentation, numerous equivalents to the specific procedures described herein. Such equivalents are considered to be within the scope of the invention. Various substitutions, alterations, and modifications may be made to the invention without departing from the spirit and scope of the invention. Other aspects, advantages, and modifications are within the scope of the invention. The contents of all references, issued patents, and published patent applications cited throughout this application are hereby incorporated by reference. The appropriate components, processes, and methods of those patents, applications and other documents may be selected for the invention and embodiments thereof.

We claim:

1. A sensor device comprising:
a body;
an air inlet for placement proximate to a food product;
an air outlet;
at least one sensor, wherein an electrical property of the sensor varies upon exposure of the sensor to at least one of a plurality of predetermined molecules, particles, bacteria, viruses and biological cells; and
an air pump for drawing air from the inlet, across the at least one sensor and exhausting the air out the outlet.

2. The sensor device according to claim 1, wherein the body includes an ergonomic design.

3. The sensor device according to claim 1, wherein the device comprises a plurality of sensors.

4. The sensor device according to claim 1, wherein the at least one sensor is provided on a replaceable sensor card.

5. The sensor device according to claim 3, further comprising a slot for receiving a replaceable sensor card.

6. The sensor device according to claim 4, wherein the replaceable sensor card includes a plurality of sensors.

7. The sensor device according to claim 4, wherein the replaceable sensor card includes a memory.

8. The sensor device according to claim 1, further comprising a microcontroller.

9. The sensor device according to claim 1, further comprising visual and/or audible means for aiding in operating the device and/or presenting a test result.

10. The sensor device according to claim 9, wherein the visual means comprises one or more LED lights.

11. The sensor device according to claim 9, wherein the audible means comprises a piezo buzzer.

12. The sensor device according to claim 1, wherein the electrical property comprises at least one of resistance and capacitance.

13. The sensor device according to claim 1, wherein the air pump comprises a motor and fan.

14. A sensor device comprising:
an ergonomic body including a slot for receiving a sensor card;
a microcontroller;
a plurality of LEDs for displaying operational status of the sensor device and/or displaying a result;
audio means for audibly presenting operation status of the sensor device and/or audibly presenting a result;
a control program operational on the microcontroller for operating the sensor device and for determining a freshness of food;
a first air inlet for placement proximate to a food product to receive air from around a food product for testing;
a replaceable sensor card including a plurality of sensors an a sensor card inlet and a sensor card outlet, wherein the sensor card receives air in the sensor card inlet from the first air inlet, the air being vented out of the sensor card via sensor card outlet, wherein a resistance of each sensor varies upon exposure of the sensor to at least one of a plurality of predetermined molecules, particles, bacteria, viruses and biological cells; and
a fan assembly including a manifold having an outlet for exhausting air from the fan assembly and/or the sensor device, a fan rotor and a cover, the cover including a fan assembly inlet which receives air from the sensor card outlet, the received air being exhausted via the outlet in the manifold.

15. A replaceable sensor card for a sensor device comprising:
a plurality of sensors;
a plurality of electrical contacts connected with the plurality of sensors, the contacts being received in a corresponding electrical connectors in a sensor device;
a sensor card inlet; and
a sensor card outlet, wherein the sensor card receives air into the sensor card via the inlet, the air eventually being vented out of the sensor card via sensor card outlet, wherein a resistance of each sensor varies upon exposure of the sensor to at least one of a plurality of predetermined molecules, particles, bacteria, viruses and biological cells.

16. A method for determining freshness of a food product comprising:
measuring a first value of one or more electrical properties of one or more sensors in a food sensor device prior to the one or more sensors being exposed to an air flow received from adjacent a food product for testing;
exposing the one or more of the sensors to a the air flow;
measuring a second value of the one or more electrical properties of the one or more sensors;
determining if the second value is greater, by a predetermined amount, than the first value.

17. The method for determining freshness of a food product according to claim 16, wherein upon the second value being greater by the predetermined amount than the first value, the method further comprises presenting a visual and/or audio indication that the food product is spoiled.

18. The method for determining freshness of a food product according to claim 16, wherein upon the second value being equal to or less than the first value, the method further comprises presenting a visual and/or audio indication that the food product is fresh.

19. The method for determining freshness of a food product according to claim 16, wherein upon the second value being greater than the first value, but less the predetermined amount greater than the first value, the method further comprises the step of presenting a visual and/or audio indication that the food product is approaching spoilage.

20. The method for determining freshness of a food product according to claim 16, further comprising determining a number of sensors in which the second value for a corresponding sensor is greater, by the predetermined amount, then the first value for the respective sensor.

21. The method according to claim 20, wherein upon the number of sensors being greater than a majority, the method further comprises the step of presenting a visual and/or audible indication that the food product is spoiled.

22. The method for determining freshness of a food product according to claim 18, further comprising determining a number of sensors in which the second value is equal to or less than the first value.

23. The method according to claim 22, wherein upon the number of sensors being greater than a majority, the method further comprises the step of presenting a visual and/or audible indication that the food product is fresh.

24. The method for determining freshness of a food product according to claim 18, further comprising determining a number of sensors in which the second value of each sensor is greater than the first value, but less the predetermined amount greater than the first value

25. The method according to claim 24, wherein upon the number being greater than a majority, the method further comprises the step of presenting a visual and/or audio indication that the food product is approaching spoilage.

26. A system for determining freshness of a food product comprising:
measuring means for measuring at least one of:
a first value of one or more electrical properties of one or more sensors in a food sensor device prior to the one or more sensors being exposed to an air flow received from adjacent a food product for testing; and
a second value of the one or more electrical properties of the one or more sensors;
exposing means for exposing the one or more of the sensors to a the air flow; and
determining means for determining if the second value is greater, by a predetermined amount, than the first value.

27. A method for determining freshness of a food product comprising:
measuring a first value of one or more electrical properties of one or more sensors in a food sensor device prior to the one or more sensors being exposed to an air flow received from adjacent a food product for testing;
exposing the one or more of the sensors to a the air flow;
measuring a second value of the one or more electrical properties of the one or more sensors;
purging the air from around the one or more sensors;
measuring a third value of the one or more electrical properties of the one or more sensors;
comparing the measured values for each of the one or more sensors to determine the freshness of the food product;
determining an individual freshness result of each of the one or more sensors based on the comparison;
determining an ultimate freshness result of the food product based on the individual freshness results of each of the one or more sensors; and
presenting a visual and/or audio indication of the freshness of the food product.

28. The method according to claim 27, wherein the individual freshness results comprise one of three food states: fresh, approaching spoilage, spoilage.

29. The method according to claim 28, wherein determining the ultimate freshness result comprises adding the number of fresh results, approaching spoilage results and spoilage results.

30. The method according to claim 29, wherein the ultimate freshness result comprises spoilage upon a majority of the individual sensor results indicate spoilage.

31. The method according to claim 29, wherein the ultimate freshness result comprises approaching spoilage upon a majority of the individual sensor results indicate approaching spoilage.

32. The method according to claim 29, wherein the ultimate freshness result comprises fresh if a substantial majority of the individual sensor results indicate fresh.

33. The method according to claim 29, wherein adding comprises using one or more counters to count a result of each food state.

34. A system for determining freshness of a food product comprising:
measuring means for measuring at least one of:
a first value of one or more electrical properties of one or more sensors in a food sensor device prior to the one or more sensors being exposed to an air flow received from adjacent a food product for testing;
a second value of the one or more electrical properties of the one or more sensors after exposure of the sensors to an air flow obtained from around a food product for testing; and
a third value of the one or more electrical properties of the one or more sensors;
exposing means for exposing the one or more of the sensors to a the air flow;
purging means for purging air from around the one or more sensors;
comparing means for comparing the measured values for each of the one or more sensors to determine the freshness of the food product; and
determining means for determining at least one of:
an individual freshness result of each of the one or more sensors based on the comparison; and
an ultimate freshness result of the food product based on the individual freshness results of each of the one or more sensors; and
presenting means for presenting a visual and/or audio indication of the freshness of the food product.

35. A method for manufacturing a material having variable electrical properties upon exposure to at least one of particles, molecules and biological cells comprising:
dissolving or suspending polyaniline in a solvent;
ultrasonically agitating the polyaniline-solvent mixture;
combining the ultrasonically agitated polyaniline-solvent mixture with an acid; and
combining the acid-polyaniline-solvent mixture with carbon.

36. The method according to claim 35, further comprising ultrasonically agitating the carbon-acid-polyaniline-solvent mixture.

37. The method according to claim 35, wherein ultrasonic agitation is applied between about five (5) minutes and about thirty (30) minutes.

38. The method according to claim 35, wherein ultrasonic agitation is applied between about ten (10) minutes and about twenty (20) minutes.

39. The method according to claim 35, wherein ultrasonic agitation is applied for about 15 minutes.

40. The method according to claim 36, wherein ultrasonic agitation is applied between about five (5) minutes and about thirty (30) minutes.

41. The method according to claim 36, wherein ultrasonic agitation is applied between about ten (10) minutes and about twenty (20) minutes.

42. The method according to claim 36, wherein ultrasonic agitation is applied for about 15 minutes.

43. A sensor material capable of variable electrical properties upon exposure to particles, molecules and/or biological cells contained in an airflow, comprising:
between about 35-70 weight percent polyaniline;
between about 5-25 weight percent carbon; and
between about 15-86 weight percent carbon.

44. The sensor material according to claim 43, wherein the polyaniline weight percent is between about 45-60 weight percent, the carbon is between about 10-20 weight percent, and the acid is between about 25-60 weight percent.

45. The sensor material according to claim 43, wherein the polyaniline weight percent is about 56 weight percent, the carbon is about 14 weight percent, and the acid is about 30 weight percent.

46. A method of attenuating resistance drift in a conductive polymer, comprising exposing the conductive polymer to at least one of ammonia gas, amine vapors and spoiled food vapors.

47. The method according to claim 46, wherein the conductive polymer is exposed to the gas and/or vapors for a predetermined period of time.

48. The method according to claim 47, wherein the predetermined period of time comprises between about 1 second and about 60 seconds.

49. The method according to claim 47, wherein the predetermined period of time comprises between about 1 second and about 30 seconds.

50. The method according to claim 47, wherein the predetermined period of time comprises about 10 seconds.

51. The method according to claim 46, wherein prior to exposing the conductive polymer to gas and/or vapors, the method comprises applying the polymer to a ceramic substrate.

52. The method according to claim 51, wherein the polymer is subjected to drying for a period of time on the ceramic substrate prior to exposure to the gas and/or vapors.

53. The method according to claim 52, wherein the period of time comprises about one hour and about 10 hours.

54. The method according to claim 52, wherein the period of time comprises about 3 hours and about 8 hours.

55. The method according to claim 52, wherein the period of time comprises about 7 hours.
